(19) United States
(12) Patent Application Publication
(10) Pub. No.: US 2022/0088309 A1
(43) Pub. Date: Mar. 24, 2022

(54) AUTOMATIC INJECTION DEVICE

(71) Applicant: AbbVie Inc., North Chicago, IL (US)

(72) Inventors: Sherwin S. Shang, Vernon Hills, IL (US); Esra Ozdaryal, Deerfield, IL (US); Eduard N. Tsvirko, Arlington Heights, IL (US); Edwin Chim, Vernon Hills, IL (US); David A. Post, Kenosha, WI (US); William P. Szechinski, Chicago, IL (US); Shubha Chethan Somashekar, Waukegan, IL (US); Ryan Brumund, Libertyville, IL (US)

(21) Appl. No.: 17/306,815

(22) Filed: May 3, 2021

Related U.S. Application Data
(63) Continuation-in-part of application No. 15/782,751, filed on Oct. 12, 2017, now abandoned.
(60) Provisional application No. 62/407,254, filed on Oct. 12, 2016.

(51) Int. Cl. A61M 5/20; A61M 5/315

(52) U.S. Cl. CPC A61M 5/2033; A61M 2207/00; A61M 5/315

(57) ABSTRACT

Systems, methods, and devices are disclosed for facilitating injection of a medicament using an automatic injection device. The automatic injection device includes a housing defining a confined inner space and having a length extending from a proximal end to a distal end along a longitudinal axis. A helical biasing member, or spring, is disposed in the confined inner space, and the spring has an inner diameter at a middle portion that is greater than an inner diameter at the terminal ends of the spring. A syringe plunger has a second bifurcated end extending into an inner bore of the spring. The bifurcated end includes two flexible arms that are able to flex inwardly and outwardly relative to the longitudinal axis within the inner bore of the spring while maintaining an annular gap between the syringe plunger and the spring.

### CROSS-REFERENCE TO RELATED APPLICATIONS

This application is a continuation-in-part of and claims the benefit of priority to U.S. application Ser. No. 15/782,751, filed Oct. 12, 2017, which claims the benefit of priority to U.S. Provisional Application No. 62/407,254, filed Oct. 12, 2016, both applications are incorporated by reference in their entirety.

### TECHNICAL FIELD

The present disclosure relates to an automatic injection device for injecting a substance, such as a medicament, into a patient.

### BACKGROUND

One of the most common routes of administration for medicaments is by injection, such as intravenous, subcutaneous or intramuscular injection. A syringe containing a medicament is used for the injection, which is often carried out by trained medical personnel. In certain instances, a patient is trained in the use of the syringe to allow for self-injection. Moreover, certain medicaments are formulated in pre-filled syringes for patient use, to avoid the need for the patient to fill the syringe. Some patients, however, may be averse to carrying out self-injection, particularly if the patient has a fear of needles. Automatic injection devices offer an alternative to a syringe for delivering a medicament, as the needle is shielded to help prevent accidental sticks and conceal the needle from view by the patient.

### SUMMARY

The present disclosure provides improved automatic injection devices, components thereof, and methods of administering an injectable medicament to a patient.

In an embodiment, the present disclosure provides an automatic injection device having a distal end configured to deliver a medicament held in a container therein and a proximal end configured to be controllable by a user. The automatic injection device includes a housing defining a confined inner space of the automatic injection device, the housing having a length extending from the proximal end to the distal end along a longitudinal axis. The automatic injection device includes a helical biasing member disposed in the confined inner space of the housing along the longitudinal axis having an inner bore and a length extending from a first terminal end of the helical biasing member to a second terminal end of the helical biasing member opposite the first terminal end. The helical biasing member also includes an inner diameter at a middle portion between the first terminal end and the second terminal end having a first inner diameter greater than a second inner diameter at the first terminal end or at the second terminal end.

The automatic injection device includes a syringe plunger having a first end extending into the container and a second bifurcated end extending into the inner bore of the helical biasing member along the longitudinal axis. The bifurcated end of the syringe plunger has a first flexible arm and a second flexible arm, the first arm having a first projection at a first end thereof and the second arm having a second projection at a first end thereof. The first and the second flexible arms are able to flex inwardly and outwardly relative to the longitudinal axis within the inner bore of the helical biasing member while maintaining an annular gap between the syringe plunger and the helical biasing member.

In accordance with embodiments of the present disclosure, a method of forming an automatic injection device is disclosed. The automatic injection device has a distal end configured to deliver a medicament held in a container therein and a proximal end configured to be controllable by a user. The method includes providing a housing defining a confined inner space of the automatic injection device. The housing has a length extending from the proximal end to the distal end along a longitudinal axis. The method includes providing a helical biasing member disposed in the confined inner space of the housing along the longitudinal axis. The helical biasing member has an inner bore and a length extending from a first terminal end of the helical biasing member to a second terminal end of the helical biasing member opposite the first terminal end. The helical biasing member also has an inner diameter at a middle portion between the first terminal end and the second terminal end having a first inner diameter greater than a second inner diameter at the first terminal end or at the second terminal end.

The method includes providing a syringe plunger having a first end extending into the container and a second bifurcated end extending into the inner bore of the helical biasing member along the longitudinal axis. The bifurcated end of the syringe plunger has a first flexible arm and a second flexible arm, the first arm having a first projection at a first end thereof and the second arm having a second projection at a first end thereof. The first and the second flexible arms are able to flex inwardly and outwardly relative to the longitudinal axis within the inner bore of the helical biasing member while maintaining an annular gap between the syringe plunger and the helical biasing member.

In accordance with some embodiments of the present disclosure, a method of forming an automatic injection device is disclosed to reduce the occurrence of a wet injection. The method includes providing an automatic injection device having a distal end configured to deliver a medicament held in a container therein and a proximal end configured to be controllable by a user. The method also includes providing a housing having an inner surface defining a confined inner space of the automatic injection device. The housing has a length extending from the proximal end to the distal end along a longitudinal axis and a radial stop extending radially inward from a distal end of the inner surface. The method also includes providing a helical biasing member disposed in the confined inner space of the housing along the longitudinal axis having an inner bore and a length extending from a first terminal end of the helical biasing member to a second terminal end of the helical biasing member opposite the first terminal end. The helical biasing member has an inner diameter at a middle portion between the first terminal end and the second terminal end with a first inner diameter greater than a second inner diameter at the first terminal end or at the second terminal end.

The method includes providing a syringe plunger having a first end extending into the container and a second bifurcated end extending into the inner bore of the helical biasing member along the longitudinal axis. The bifurcated end of the syringe plunger has a first flexible arm and a second flexible arm, the first arm having a first projection at a first end thereof and the second arm having a second projection at a first end thereof. The first and the second flexible arms are able to flex inwardly and outwardly relative to the longitudinal axis within the inner bore of the helical biasing member while maintaining an annular gap between the syringe plunger and the helical biasing member. The method includes engaging the first projection and the second projection with the radial stop to maintain the syringe plunger in a latched position. The method includes providing a firing button including an inner ring configured to disengage the first projection and the second projection from the radial stop when the firing button is activated by the user. When the firing button is activated, the first and second flexible arms are able to flex inwardly and outwardly relative to the longitudinal axis centrally located within the housing without contacting the helical biasing member. As such, the helical biasing member is able to bias the syringe and syringe plunger toward the distal end of the device without loss of force to reduce the occurrence of a wet injection.

### BRIEF DESCRIPTION OF THE FIGURES

The foregoing and other objects, features and advantages of the exemplary embodiments will be more fully understood from the following description when read together with the accompanying drawings, in which:

FIG. 1 illustrates an exemplary automatic injection device suitable for injecting a dose of a medicament according to an example embodiment.

FIG. 2 illustrates another exemplary automatic injection device suitable for injecting a dose of a medicament according to an example embodiment.

FIG. 3 is an exploded view of a firing mechanism assembly, according to an example embodiment.

FIG. 4 is an exploded view of another firing mechanism assembly, according to an example embodiment.

FIG. 5A is a side view of an exemplary plunger of the firing mechanism assembly of FIG. 3 and FIG. 4, according to an example embodiment.

FIG. 5B is a side view of another exemplary plunger suitable for use in the automatic injection devices taught herein.

FIG. 6 is a side view of a helical biasing member, according to an exemplary embodiment.

FIG. 7 is a perspective view of the helical biasing member of FIG. 6, according to an exemplary embodiment.

FIG. 8 is an end view of the helical biasing member of FIG. 6 looking through the inner bore of the helical biasing member, according to an exemplary embodiment.

FIG. 9 is a cross-sectional view of a plunger, proximal end of an automatic injection device, and a conventional biasing member, according to an exemplary embodiment.

FIG. 10 is a cross-sectional view of the plunger, proximal end of the automatic injection device, and a helical biasing member, according to an exemplary embodiment.

FIG. 11 is another cross-sectional view of the plunger, proximal end of the automatic injection device, and the conventional biasing member of FIG. 9, according to an exemplary embodiment.

FIG. 12 is another cross-sectional view of the plunger, proximal end of the automatic injection device, and the helical biasing member of FIG. 10, according to an exemplary embodiment.

FIG. 13 is another cross-sectional view of the plunger, proximal end of the automatic injection device, and the conventional biasing member of FIG. 9, according to an exemplary embodiment.

FIG. 14 is another cross-sectional view of the plunger, proximal end of the automatic injection device, and the helical biasing member of FIG. 10, according to an exemplary embodiment.

FIG. 15 is another cross-sectional view of the plunger, proximal end of the automatic injection device, and the conventional biasing member of FIG. 9, according to an exemplary embodiment.

FIG. 16 is another cross-sectional view of the plunger, proximal end of the automatic injection device, and the helical biasing member of FIG. 10, according to an exemplary embodiment.

FIG. 17 is a perspective view of an embodiment of the proximal end of the automatic injection device of FIG. 9, according to an exemplary embodiment.

FIG. 18 is an end view of the proximal end of the automatic injection device of FIG. 17, according to an exemplary embodiment.

FIG. 19 illustrates a cross-sectional view of a portion of an automatic injection device, according to an exemplary embodiment.

FIG. 20 illustrates an example syringe carrier, according to an exemplary embodiment.

FIG. 21 is a graphical comparison of two different automatic injection devices for delivering 0.4 ml of a medicament at room temperature, according to an exemplary embodiment.

FIG. 22 is a graphical comparison of two different automatic injection devices for delivering 0.4 ml of a medicament at a refrigerated temperature, according to an exemplary embodiment.

FIG. 23 is a graphical comparison of two different automatic injection devices for delivering 0.8 mL of a medicament at room temperature, according to an exemplary embodiment.

FIG. 24 is a graphical comparison of two different automatic injection devices for delivering 0.8 mL of a solution at a refrigerated temperature, according to an exemplary embodiment.

FIG. 25 illustrates a conventional biasing member and six example helical biasing members with a barrel design, according to an exemplary embodiment.

FIG. 26A illustrates an example embodiment for a terminal end of the helical biasing member disclosed herein.

FIG. 26B illustrates another example embodiment for a terminal end of the helical biasing member disclosed herein.

FIG. 26C illustrates another example embodiment for a terminal end of the helical biasing member disclosed herein.

FIG. 26D illustrates another example embodiment for a terminal end of the helical biasing member disclosed herein.

FIG. 27 illustrates a cross-sectional view of a portion of an automatic injection device, according to an exemplary embodiment.

FIG. 28 illustrates a cross-sectional view of a portion of a syringe carrier and a syringe lockout shroud, according to an exemplary embodiment.

FIG. 29 illustrates another cross-sectional view of a portion of a syringe carrier and a syringe lockout shroud, according to an exemplary embodiment.

FIG. 30 illustrates a graphical comparison of compression forces vs distance traveled for various helical biasing members.

FIG. 31 is a flow chart illustrating a method of forming an automatic injection device, according to an exemplary embodiment.

FIG. 32 is a flow chart illustrating methods of forming an automatic injection device, according to other exemplary embodiments.

### DETAILED DESCRIPTION

The present disclosure provides automatic injection devices, components thereof, and methods for injecting a substance, such as a medicament, into a patient. A housing of an automatic injection device defines an inner space, within which a helical biasing member is disposed. When the automatic injection device is actuated, the helical biasing member drives a plunger toward a distal end of the device to initiate an injection of the medicament from a syringe. The helical biasing member is designed such that the inner diameter of the coils at its middle portion is greater than the inner diameter of the coils at its terminal ends. This design prevents the helical biasing member from buckling when compressed, and prevents undesirable interactions between the helical biasing member and the plunger during operation of the automatic injection device. The avoidance of the undesirable interaction between the helical biasing member and the plunger during operation avoids a temporary loss of force on the plunger while dispensing the medicament. The ability of the helical biasing member to maintain force on the plunger is one solution to an undesirable effect known as a “wet injection.”

The apparatus and methods presented herein can be used for injecting a variety of medicaments into a patient. In one embodiment, the automatic injection device can be configured in the form of a pen, i.e., a portable autoinjector that enables an individual to administer a dosage of a medicament.

The helical biasing member is also designed to provide a stronger force that may shorten delivery times (e.g. to less than 15 seconds) and also may effectively deliver medicaments having viscosities between about 0.05 to about 50 centipoise. However, increasing the expansion force of the helical biasing member may introduce additional challenges, such as increasing the probability of a glass syringe breaking under the expansion force of the helical biasing member.

As used herein, an “automatic injection device” (or “autoinjector”) is intended to refer to a device that enables an individual (also referred to herein as a user or a patient) to self-administer a dosage of a medicament. The automatic injection device differs from a standard syringe by the inclusion of a mechanism for automatically inserting the needle at an injection site, delivering the medicament to the individual by injection, and retracting the needle from the injection site when the mechanism is engaged.

As used herein, the term “medicament” refers to a composition intended for use in medical diagnosis, cure, treatment, or prevention of disease. A medicament may be a therapeutic agent or a combination of therapeutic agents. A medicament may include a therapeutic protein, for example, a peptide or antibody, or antigen-binding portion thereof. A medicament may include an anesthetic, steroid, and/or any other therapeutic agent(s). In one embodiment, a medicament represents a mixture of two or even more pharmacologically active agents. In some embodiments, the medicament is a liquid therapeutic agent which includes one or more biological agents, such as a protein, or antibody. For example, one such liquid therapeutic agent may comprise an antibody drug conjugate (ADC).

As used herein, the term “proximal” refers to the portion or end of an automatic injection device or component in the automatic injection device furthest from an injection site of the user when the device is held against the person for an injection.

As used herein, the term “distal” refers to the portion or end of an automatic injection device or a component of the automatic injection device closest to an injection site of the user during an injection.

The present disclosure provides automatic injection devices, components thereof, and methods for facilitating injection of a medicament while reducing wet injection events. A wet injection event occurs when a portion of the medicament intended to be injected into the patient is found on the skin on or near the injection site, and can range from a few drops to pooling of the medicament at the injection site. Wet injection events can occur for a number of reasons, including user error. User error can occur when the user does not hold the automatic injection device firmly against the injection site. Wet injection events can also occur when the automatic injection device begins retracting the needle from the injection site while still injecting a medicament contained in the syringe.

It has been discovered by the inventors that a wet injection event can be attributed, at least in part, to a temporary loss of force applied to a syringe plunger by a conventional biasing member during injection. As taught herein, the temporary loss of force applied to the syringe is solved without increasing the amount of constrained space within an automatic injection device. As taught herein, an undesirable interaction between projections of a syringe plunger of the automatic injection device and a conventional biasing member during injection causes the temporary loss of force on the syringe plunger. Another undesirable effect of the interaction of a conventional biasing member and the projections of the syringe plunger is the altering of an injection trajectory of the syringe plunger away from a center line of the automatic injection device along a longitudinal axis of movement.

As taught herein, a middle portion of a helical biasing member has an inner diameter larger than inner diameters of end portions thereof e.g. having a barrel like shape. The larger inner diameter of the middle portion avoids the undesirable interaction with protrusions located at a terminal end of a syringe plunger during injection, even though the end portions of the helical biasing member have an inner diameter less than that of the middle portion. The helical biasing member, as taught herein, avoids the temporary loss of force on the syringe plunger attributable to wet injection events. The helical biasing member, as taught herein, also solves the altering of the syringe trajectory during injection. The larger inner diameter allows the syringe plunger to track the center line of the automatic injection device during injection.

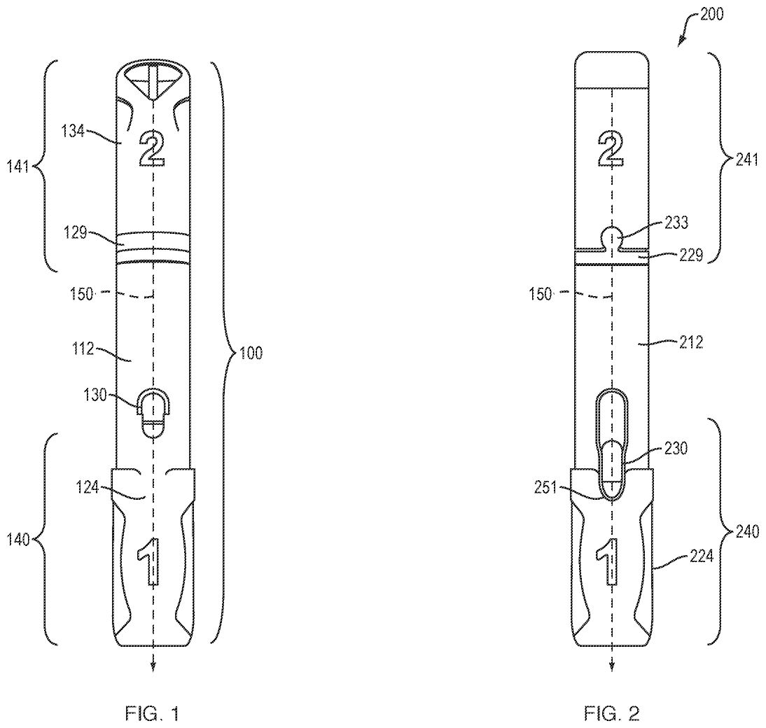

FIG. 1 illustrates an exemplary automatic injection device 100 suitable for injecting a dose of a medicament into a patient according to an example embodiment. The automatic injection device 100 includes a housing 112 for housing a container, such as a syringe, containing a dose of a medicament. The automatic injection device 100 includes a distal end 140 for placing at an injection site to deliver the medicament held in the container and a proximal end 141 for gripping by a user. The automatic injection device 100 may include a first removable cap 124, or needle cap, for covering a portion of the distal end 140 of the housing 112 to prevent exposure of the needle of the syringe prior to use. A second removable cap 134, or actuator cap, may cover a portion of the proximal end 141 of the housing 112 to prevent accidental actuation of an activation button. In some embodiments, the housing 112 may include a protrusion or step 129 extending radially outward from the exterior of the housing 112 to facilitate seating of the second removable cap 134 on the proximal end 141 of the housing 112. The housing 112 also may include a display window 130 to allow a user to view the contents of the syringe housed within the housing 112. The window 130 may include an opening in the sidewall of the housing 112, or may include a translucent material in the housing 112 to allow viewing of the interior of the device 100.

In some embodiments, the housing 112 may be a single unitary piece, while in other embodiments the housing may include multiple housing components. For example, a distal housing component and a proximal housing component can be joined together to form the housing 112 using a fastening mechanism. The fastening mechanism can include, for example, a threaded portion that allows the two components to be screwed together, one or more tabs or protrusions that may snap-fit into corresponding openings in one or both of the components, or any other fastening mechanism suitable for adhering the housing components together.

The housing 112 may have a tubular configuration, though one skilled in the art will recognize that the housing 112 may have any suitable size, shape or configuration for housing a syringe or other container of a medicament to be injected.

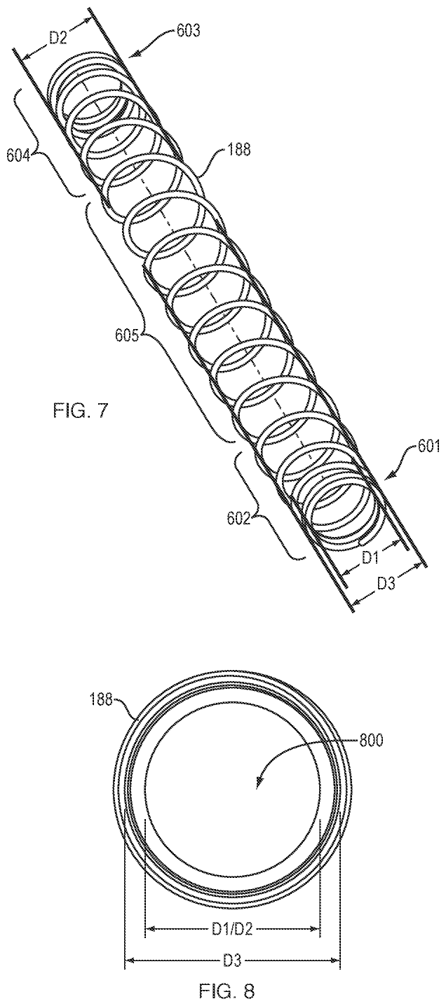

While the disclosure will be described with respect to a syringe, one skilled in the art will recognize that the automatic injection device 100 may employ any suitable container for storing and dispensing a medicament, for example, an ampoule or cartridge. The syringe (not shown in FIG. 7) may be slidably mounted within the housing 112, as described in detail below, and capable of moving within the housing 112 along a longitudinal axis 150. Prior to the automatic injection device being activated, the syringe is sheathed and retracted within the housing 112. When the automatic injection device is actuated, a needle of the syringe projects from the distal end 140 of the housing 112 to allow injection of a medicament from the syringe into a patient at an injection site. After an injection is completed, the syringe retracts within the automatic injection device 100 and the needle no longer projects from the distal end 140 of the housing. The housing 112 may be formed of any suitable surgical material, including, but not limited to, plastic and other known materials.

The second removable cap 134 may have a distinctive color to differentiate the distal end 140 and the proximal end 141 of the device. In some embodiments, the housing 112 and caps 124 and 134 may further include graphics, symbols, and/or numbers to facilitate use of the automatic injection device 100. For example, in the illustrative embodiment shown in FIG. 1, the first removable cap 124 is labeled with a “1” to indicate that a user should remove the first removable cap 124 of the device first. The second removable cap 134 is labeled with a “2” to indicate that the second removable cap 134 should be removed after the first removable cap 124 is removed. One skilled in the art will recognize that the automatic injection device 100 may have any suitable graphics, symbols and/or numbers to facilitate user instruction, or the automatic injection device may omit such graphics, symbols and/or numbers.

FIG. 2 illustrates another exemplary automatic injection device 200 suitable for injecting a dose of a medicament into a patient according to an example embodiment. The automatic injection device 200 includes a housing 212 for housing a container, such as a syringe, containing a dose of a medicament. The syringe (not shown in FIG. 7) may be slidably mounted within the housing 212 and capable of moving within the housing 212 along a longitudinal axis 150. The automatic injection device 200 includes a distal end 240 for placing at an injection site to deliver a medicament held in the container and a proximal end 241 for gripping by a user. The automatic injection device 200 may include a first removable cap 224, or needle cap, for covering a portion of the distal end 240 of the housing 212 to prevent exposure of the needle of the syringe prior to use. A second removable cap 234, or actuator cap, may cover a portion of the proximal end 241 of the housing 212 to prevent accidental actuation of an activation button. In some embodiments, the housing 212 may include a protrusion or step 229 extending radially outward from the exterior of the housing 212 to facilitate seating of the second removable cap 234 on the proximal end 241 of the housing 212. The housing 212 also may include an elongated display window 230 to allow a user to view the contents of the syringe housed within the housing 212. The window 230 may include an opening in the sidewall of the housing 212, or may include a translucent material in the housing 212 to allow viewing of the interior of the device 200.

The first removable cap 224 can include a notch 251 to align with a portion of the elongated window 230 to prevent obstruction of the window 230 when the first removable cap 224 is positioned on the housing 212. The proximal end 241 of the housing 212 can include one or more mating tabs 233 extending from the step 229, in some embodiments, that can be configured to mate with one or more receptacles or cut-out portions of the second removable cap 234. For example, the one or more mating tabs 233 can snap-fit into a portion of the second removable cap 234 and lock the second removable cap 234 to the proximal end 241 of the housing 212 and prevent inadvertent removal of the second removable cap 234. The one or more mating tabs 233 can also align the second removable cap 234 with the housing 212 during assembly and prevent rotation of the second removable cap 234 relative to the housing 212 during transportation or handling of the automatic injection device 200, which can prevent accidental firing of the automatic injection device 200.

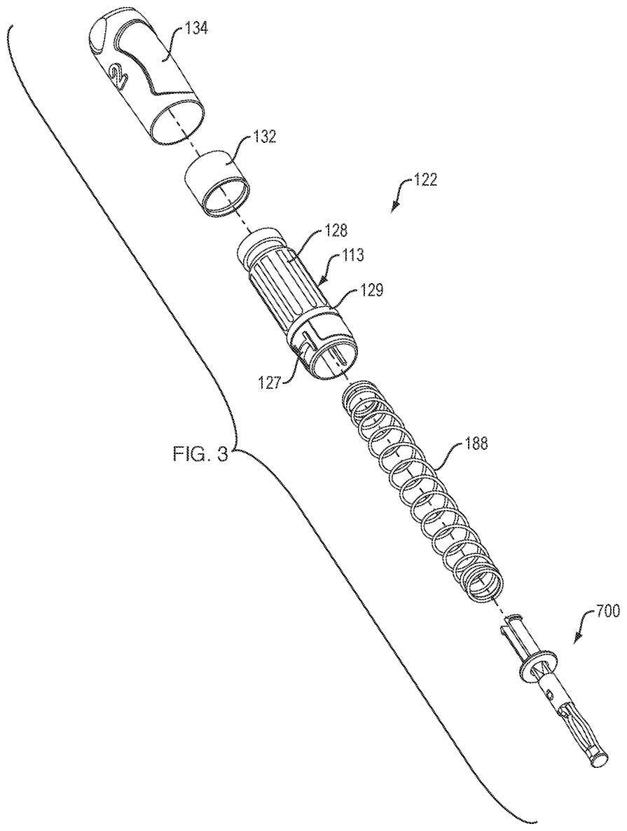

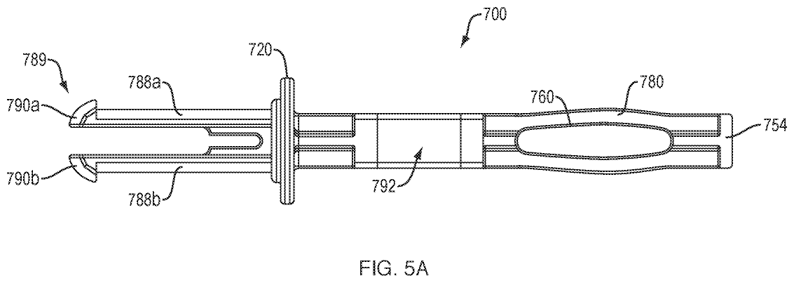

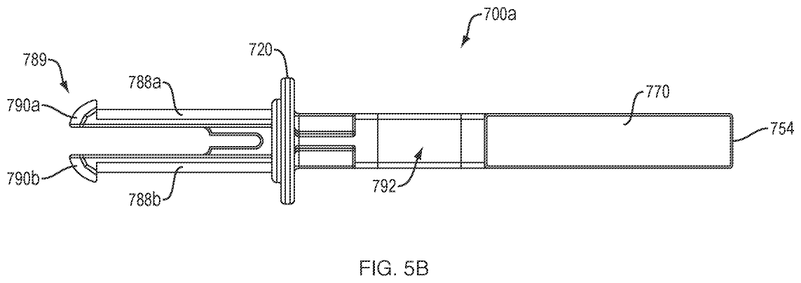

FIG. 3 is an exploded view of a firing mechanism assembly 122, according to an exemplary embodiment. The firing mechanism assembly 122 is located at the proximal portion 141 of the automatic injection device 100. As shown, the firing mechanism assembly 122 includes a firing button, or activation button 132, a gripping region 113 of the housing, and a helical biasing member 188. The gripping region 113 may be a unitary part of the housing 112, discussed above in reference to FIG. 1, or may be formed of a separate tubular member matable to another tubular member. As will be discussed in more detail below, the helical biasing member 188 has a barrel design, and therefore the inner diameter of the coils at the middle portion of the helical biasing member 188 is greater than the inner diameter of the coils at each end. The illustrative firing mechanism assembly 122 also includes a syringe plunger 700 for moving a syringe under the force of the helical biasing member 188 and actuating the syringe to expel its contents. The details of the syringe plunger 700 are discussed below in relation to FIG. 5. The gripping region 113 can also include contours 128, in some embodiments, to facilitate gripping of the device once the second removable cap 134 has been removed. The gripping region 113 also includes the step 129 formed, in some embodiments, in the distal portion of the gripping region 113 to facilitate seating of the second removable cap 134.

The gripping region 113 generally has a tubular configuration, though one skilled in the art will recognize that the gripping region 113 can have any number of suitable shapes and configurations for housing a syringe or other container of a medicament to be injected. In exemplary embodiments, the gripping region 113 is a proximal component of the housing 112 of the automatic injection device 100, discussed above in reference to FIG. 1, and the gripping region 113 can be coupled to a distal component of the housing 112 using a fastening mechanism. In one embodiment, the gripping region 113 can include one or more tabs 127 that may snap-fit into corresponding openings on a distal component of housing 112 to ensure alignment and coupling of the components.

The activation button 132 can be used for actuating the automatic injection device by releasing the plunger 700 from a resting position and allowing the helical biasing member 188 to propel the plunger 700 toward the distal end of the automatic injection device and drive a syringe forward such that the syringe needle projects from the distal end of the automatic injection device, pierces the skin of the user at the injection site, and the medicament within the syringe is expelled through the needle into the patient.

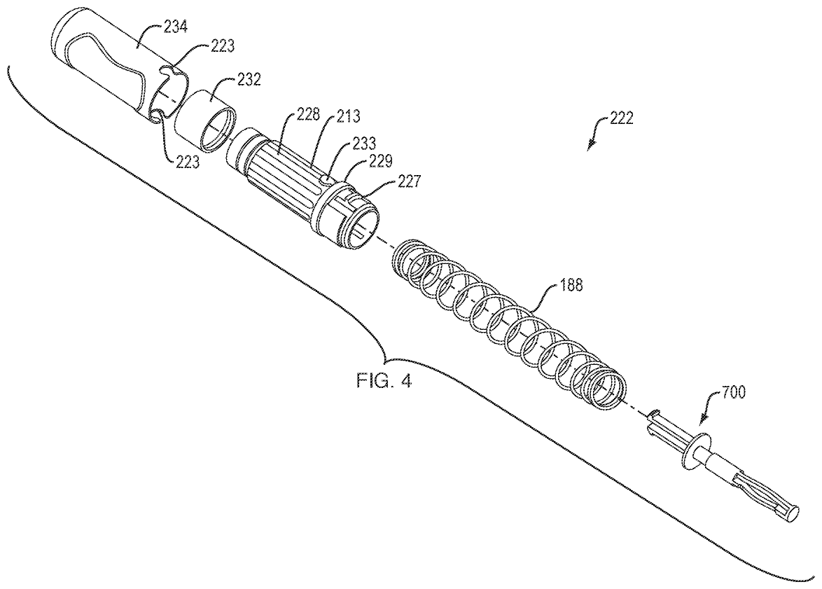

FIG. 4 is an exploded view of another firing mechanism assembly 222, according to an exemplary embodiment. The firing mechanism assembly 222 is located at the proximal portion 241 of the automatic injection device 200. As shown, the firing mechanism assembly 222 includes a firing button or activation button 232, a gripping region 213 of the housing, and the helical biasing member 188. The gripping region 213 may be a unitary part of the housing 212, discussed above in reference to FIG. 2, or may be formed of a separate tubular member matable to another tubular member.

As will be discussed in more detail below, the helical biasing member 188 has a barrel design, and therefore the inner diameter of the coils at the middle portion of the helical biasing member 188 is greater than the inner diameter of the coils at each end. The illustrative firing mechanism assembly 222 also includes a syringe actuator, or plunger 700, for moving a syringe under the force of the helical biasing member 188 and actuating the syringe to expel its contents. The details of the syringe plunger 700 are discussed below in relation to FIG. 5. The gripping region 213 can also include contours 228, in some embodiments, to facilitate gripping of the device once the second removable cap 234 has been removed. The gripping region also includes the step 229 and one or more mating tabs 233 to facilitate seating of the second removable cap 234. The mating tabs 233 extending from the step 229 can be configured to mate with one or more receptacles 223 or cut-out portions of the second removable cap 234.

The gripping region 213 generally has a tubular configuration, though one skilled in the art will recognize that the gripping region 213 can have any number of suitable shapes and configurations for housing a syringe or other container of a medicament to be injected. In exemplary embodiments, the gripping region 213 is a proximal component of the housing 212 of the automatic injection device 200, discussed above in reference to FIG. 2, and the gripping region 213 can be coupled to a distal component of housing 212 using a fastening mechanism. In one embodiment, the gripping region 213 can include one or more tabs 227 that may snap-fit into corresponding openings on a distal component of housing 212 to ensure alignment and coupling of the components.

The activation button 232 can be used for actuating the automatic injection device by releasing the plunger 700 from a resting position and allowing the helical biasing member 188 to propel the plunger 700 toward the distal end of the automatic injection device and drive a syringe forward such that the syringe needle projects from the distal end of the automatic injection device, pierces the skin of the user at the injection site, and the medicament within the syringe is expelled through the needle into the patient.

FIG. 5A is a side view of the exemplary plunger 700 suitable for use in the automatic injection devices taught herein. In this example embodiment, the plunger 700 includes a retaining flange 720 for holding the helical biasing member 188 in a compressed position until actuation. Upon activation, the helical biasing member 188 acts upon the retaining flange 720 to drive the plunger 700 distally. The retaining flange 720 can be sized, dimensioned, and formed of a material that allows the plunger 700 to slide within the interior of the housing 112, 212 when the device is actuated. In some embodiments, the plunger 700 can be an integrated component formed of any suitable material, such as an acetal-based plastic. Extending proximally from the retaining flange 720, the plunger 700 includes a bifurcated proximal end with flexible arms 788a and 788b, around which the helical biasing member 188 is disposed in the housing 112, 212. The flexible arms 788a and 788b terminate in an anchoring portion 789, with each flexible arm 788a and 788b having respective projections 790a and 790b. The projections 790a and 790b are configured to extend radially outward beyond the flexible arms 788a and 788b, respectively, and are configured to selectively engage with an inner portion of the housing 112, 212. The anchoring portion 789 of the flexible arms 788a and 788b can include one or more angled surfaces to define a cam, or the like. For example, and as shown in FIG. 5A, the anchoring portion 789 can have a substantially arcuate shape formed by multiple edge segments, each having a different angle. Extending distally from the retaining flange 720, the plunger 700 includes a compressible portion 780 with a central opening portion 760. The plunger 700 also includes a pressurizer 754 at a distal end for applying pressure to a bung, stopper, or medicament contained in a corresponding syringe within an automatic injection device. The compressible portion 780 facilitates movement of a corresponding syringe toward an injection site and expulsion of the contents of the syringe in two separate steps, in some embodiments. The compressible portion 780 has a cross section and a length that changes during the injection process. For example, the cross sectional width of the compressible portion 780 decreases during the injection process. Likewise, a length of the compressible portion 780 increases during the injection process.

The plunger 700 can further include an indicator 792 configured to align with the elongated window 230 to indicate completion of the injection. The indicator 792 may have a distinctive color or design to represent completion of the injection. Alternatively, or in addition, the automatic injection device may include an audio or tactile indication of completion of the injection.

FIG. 5B is a side view of an exemplary plunger 700a suitable for use in the automatic injection devices taught herein. In this example embodiment, the plunger 700a lacks the compressible portion 780. In this embodiment, a rod portion 770 maintains a constant cross section and length during the entire injection operation. The plunger 700a includes a retaining flange 720 for holding the helical biasing member 188 in a compressed position until actuation. Upon activation, the helical biasing member 188 acts upon the retaining flange 720 to drive the plunger 700a distally. The retaining flange 720 can be sized, dimensioned, and formed of a material that allows the plunger 700a to slide within the interior of the housing 112, 212 when the device is actuated. In some embodiments, the plunger 700a can be an integrated component formed of any suitable material, such as an acetal-based plastic. Extending proximally from the retaining flange 720, the plunger 700a includes a bifurcated proximal end with flexible arms 788a and 788b, around which the helical biasing member 188 is disposed in the housing 112, 212. The flexible arms 788a and 788b terminate in an anchoring portion 789, with each flexible arm 788a and 788b having respective projections 790a and 790b. The projections 790a and 790b are configured to extend radially outward beyond the flexible arms 788a and 788b, respectively, and are configured to selectively engage with an inner portion of the housing 112. The anchoring portion 789 of the flexible arms 788a and 788b can include one or more angled surfaces to define a cam, or the like. For example, the anchoring portion 789 can have a substantially arcuate shape formed by multiple edge segments, each having a different angle. Extending distally from the retaining flange 720, the plunger 700a includes the rod portion 770 and a pressurizer 754 at a distal end for applying pressure to a bung, stopper, or medicament contained in a corresponding syringe within an automatic injection device. In some embodiments, the rod portion 770 of the plunger 700a can be formed in a number of different ways and can have a cross section with various geometries. For example, the rod portion 770 can be formed or molded as a solid piece of plastic. In other embodiments, the rod portion 770 can be substantially hollow with a tubular outer shape. In some embodiments, the rod portion 770 can have a cross-section that is substantially circular or cross-shaped. The rod portion 770 can also have a cross-section with a substantially equal outer diameter along its length.

The plunger 700a can further include an indicator 792 configured to align with the elongated window 230 to indicate completion of the injection. The indicator 792 may have a distinctive color or design to represent completion of the injection. Alternatively, or in addition, the automatic injection device may include an audio or tactile indication of completion of the injection.

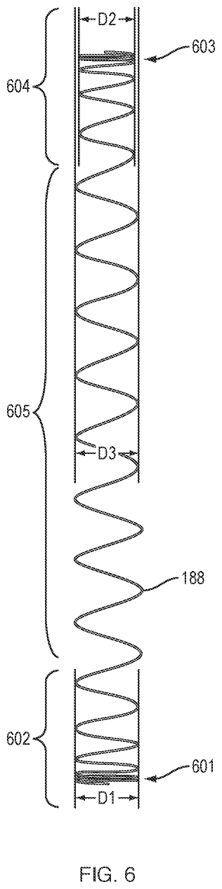

FIG. 6 is a side view of the helical biasing member 188, according to an exemplary embodiment. The helical biasing member 188 is a barrel spring having a middle portion 605 extending between a first end portion 602 and a second end portion 604. The first end portion 602 includes a first terminal end 601, and the second end portion 604 includes a second terminal end 603 opposite the first terminal end 601. The coils at the first terminal end 601 have a first inner diameter D1 that is less than the inner diameter D3 of coils at the middle portion 605 of the helical biasing member 188. Likewise, the coils at the second terminal end 603 have a second inner diameter D2 that is less than the inner diameter D3 of the coils at the middle portion 605 of the helical biasing member 188. In some embodiments, the first inner diameter D1 of the coils at the first terminal end 601 is substantially equal to the second inner diameter D2 of the coils at the second terminal end 603. When placed in an automatic injection device as taught herein, the increased inner diameter D3 of the coils at the middle portion 605, as compared to the first terminal end 601 and the second terminal end 603, reduces or eliminates buckling in the helical biasing member 188 during compression and expansion and helps maintain an annular gap between the projections 790a and 790b of the plunger 700 and the helical biasing member 188 when an automatic injection device is activated.

FIG. 7 is a perspective view of the helical biasing member 188, according to an exemplary embodiment. As discussed above, the helical biasing member 188 has a barrel design with a middle portion 605 extending between a first end portion 602 and a second end portion 604. The first end portion 602 includes a first terminal end 601, and the second end portion 604 includes a second terminal end 603 opposite the first terminal end 601. The inner diameter D1 of the coils at the first terminal end 601 is less than the inner diameter D3 of the coils at the middle portion 605 of the helical biasing member 188. Likewise, the inner diameter D2 of the coils at the second terminal end 603 is less than the inner diameter D3 of the coils at the middle portion 605 of the helical biasing member.

FIG. 8 is an end view of the helical biasing member 188 looking through an inner bore 800 extending the length of the helical biasing member 188, according to an exemplary embodiment. As can be seen in this view, the inner diameter of the coils at the first and second terminal ends 601, 603 of the helical biasing member 188 is less than the inner diameter D3 of the coils at the middle portion 605 of the helical biasing member.

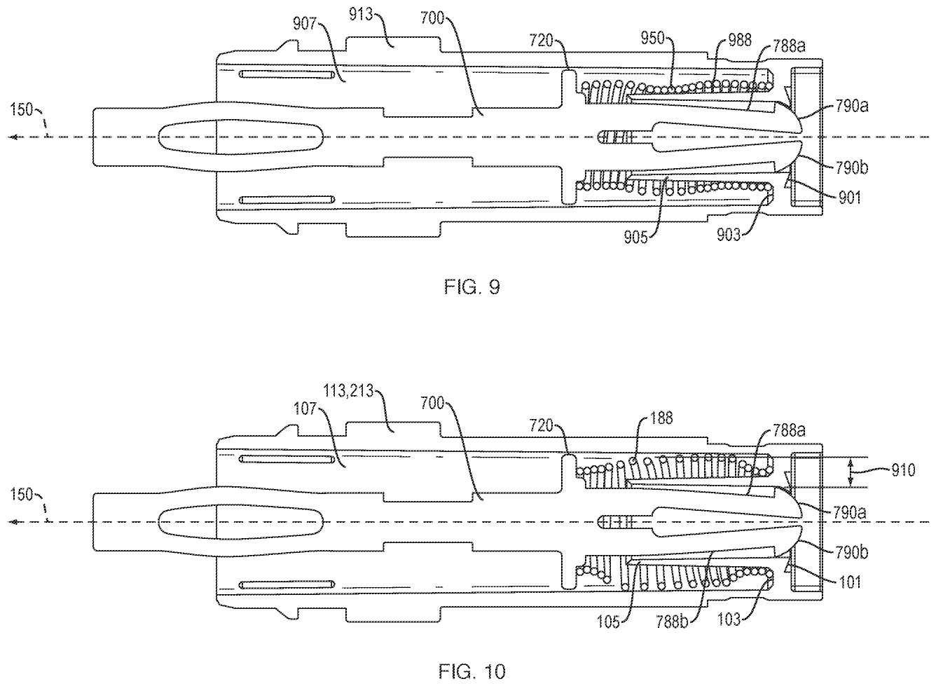

FIGS. 9-16 illustrate a comparison between a conventional automatic injection device with a conventional biasing member 988 and an automatic injection device 100, 200 with a helical biasing member 188 with a barrel design, as taught herein. FIGS. 9, 11, 13, and 15 are associated with an embodiment of the conventional automatic injection device with a conventional biasing member 988, and illustrate cross-sectional views of a portion of the device while the conventional biasing mechanism 988 is at different stages of expansion. Similarly, FIGS. 10, 12, 14, and 16 are associated with the automatic injection device 100, 200, as taught herein, and illustrate cross-sectional views of a portion of the device 100, 200 while the helical biasing member 188 is at different stages of expansion.

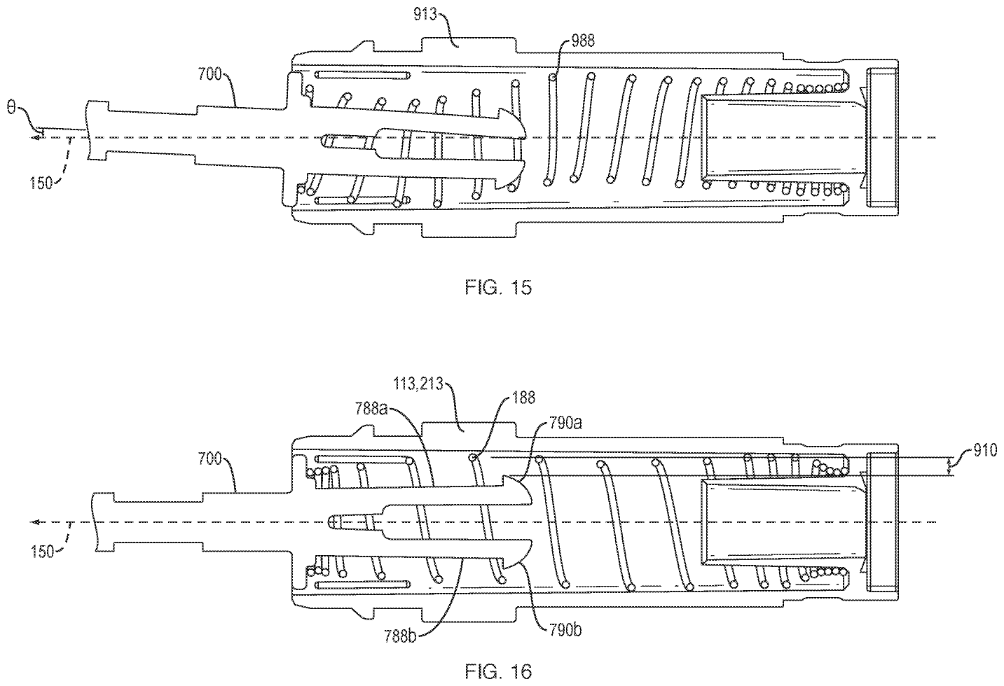

FIGS. 9-16 are shown with reference to the syringe plunger 700 having the compressible portion 780 with the central opening portion 760, as described in FIG. 5A to facilitate explanation. Nevertheless, the plungers 700 and 700a suffer from the same detrimental interaction with the conventional biasing member 988, as discussed in more detail below. The plunger 700a disclosed in FIG. 5B is well suited for use in the automatic injection device 100, 200 disclosed herein. The substitution of the syringe plunger 700 having a compressible portion 780 with the syringe plunger 700a having a non-compressible rod portion 770 may impact an offset angle θ (shown in FIG. 15) of the plunger during operation, as described in more detail below.

FIG. 9 is a cross-sectional view of the plunger 700, 700a, a gripping region 913 of an automatic injection device, and a conventional biasing member 988. The gripping region 913 includes a radial stop 901 extending radially inward from the inner surface of the gripping region 913 and configured to engage with projections 790a and 790b of the plunger. FIG. 9 illustrates a cross-sectional view of the plunger 700, 700a and conventional biasing member 988 shortly after the projections 790a and 790b have been released from engagement with the radial stop 901.

The gripping region 913 also includes an inner surface 907 and defines a confined inner space within the automatic injection device. The plunger 700, 700a and conventional biasing member 988 are disposed within the confined inner space. When the conventional biasing member 988 is compressed between the retaining flange 720 of the plunger and the radial stop 901, the projections 790a and 790b can engage with the radial stop 901 of the gripping region 913 in order to maintain the plunger 700, 700a in a latched position. In this example embodiment, the gripping region 913 includes an engagement portion 903 extending radially inward from the inner surface 907 and configured to engage with the proximal end of the conventional biasing member 988. The gripping region 913 also includes an annular collar 905 extending distally from the radial stop 901 along the longitudinal axis 150 toward the distal end of the automatic injection device within the confined inner space. In the latched position, the conventional biasing member 988 is buckled, for example, at point 950 such that a portion of the conventional biasing member 988 is pressed against the annular collar 905.

In an exemplary embodiment, an activation button (not shown) can include an inner feature or inner ring that is configured to engage with the projections 790a and 790b of the plunger 700, 700a when the activation button is depressed by a user. When the inner feature of the activation button interacts with the projections 790a and 790b, it presses the flexible arms 788a and 788b upward along the radial stop 901 conic surface toward the longitudinal axis 150 and then radially inward, towards each other, such that the projections 790a and 790b are disengaged from the radial stop 901 of the gripping region 913. Once the plunger 700, 700a is released from engagement with the gripping region 913, the conventional biasing member 988 drives the plunger 700, 700a toward the distal end of the automatic injection device. Initially, the plunger 700, 700a is driven distally along the centerline of the longitudinal axis 150 by the conventional biasing member 988.

FIG. 10 is a cross-sectional view of the plunger 700, 700a, the gripping region 113, 213 of the automatic injection device 100, 200, and the helical biasing member 188, according to an exemplary embodiment. FIG. 10 illustrates a cross-sectional view of the plunger 700, 700a and helical biasing member 188 shortly after the projections 790a and 790b have been released from engagement with the radial stop 101 of the gripping region 113, 213. The radial stop 101 extends radially upward along the radial stop 901 conic surface toward the longitudinal axis 150 and then inward from the inner surface of the gripping region 113, 213 and is configured to engage with the projections 790a and 790b.

When the helical biasing member 188 is compressed by the retaining flange 720 of the plunger 700, 700a, the projections 790a and 790b can engage with the radial stop 101 of the gripping region 113, 213 in order to maintain the plunger 700, 700a in a latched position. In this example embodiment, the gripping region 113, 213 includes an engagement portion 103 extending radially inward from the inner surface 107 and configured to engage with the terminal end 601 of the helical biasing member 188. The gripping region 113, 213 also includes an annular collar 105 extending distally from the radial stop 101 along the longitudinal axis 150 toward the distal end of the automatic injection device within the confined inner space. In contrast to the conventional biasing member 988 shown in FIG. 9, the helical biasing member 188 is not buckled in the latched position, and an annular gap 910 is maintained between the protrusions 790a and 790b of the plunger 700, 700a and the coils at least at the middle portion of the helical biasing member 188. This is because the diameter of the coils at the middle portion of the helical biasing member is greater than the diameter of the coils at the terminal ends of the helical biasing member 188.

As discussed above, when an inner feature of the activation button interacts with the projections 790a and 790b, it presses the flexible arms 788a and 788b radially inward, towards each other, such that the projections 790a and 790b are disengaged from the radial stop 101 of the gripping region 113, 213. Once the plunger 700, 700a is released from engagement with the gripping region 113, 213, the helical biasing member 188 drives the plunger 700, 700a toward the distal end of the automatic injection device 100, 200. Initially, the helical biasing member 188 drives the plunger 700, 700a distally along the centerline of the longitudinal axis 150.

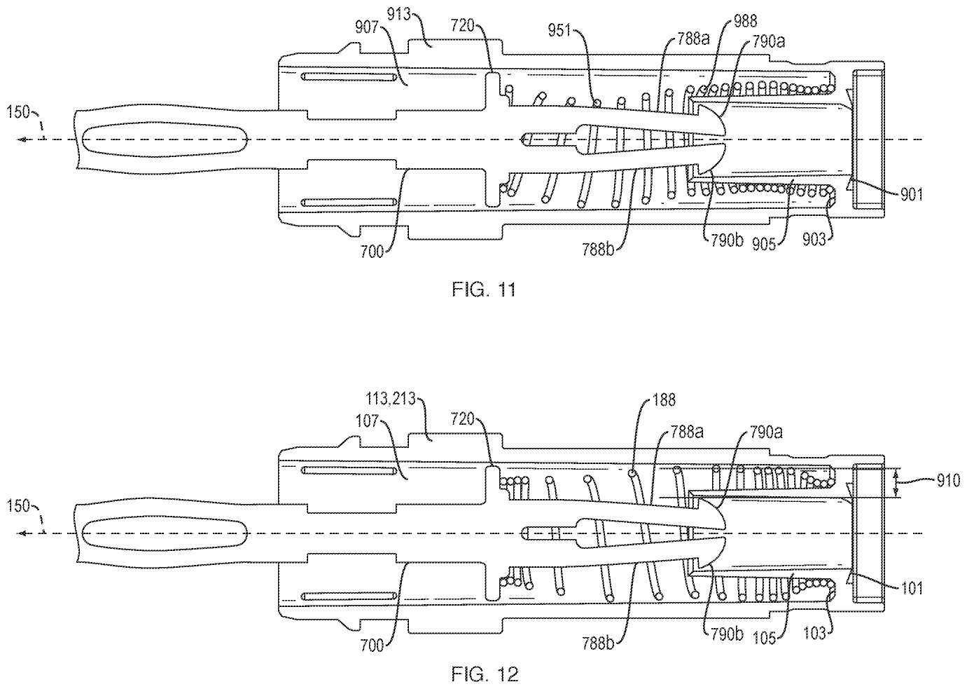

FIG. 11 is a cross-sectional view of the plunger 700, 700a, the gripping region 913, and the conventional biasing member 988 of FIG. 9 after the conventional biasing member 988 has further expanded from its compressed state. As discussed above, once the projections 790a and 790b are disengaged from the radial stop 901 of the gripping region 913, the conventional biasing member 988 drives the plunger 700, 700a toward the distal end of the automatic injection device along the longitudinal axis 150. However, since the conventional biasing member 988 is buckled within the proximal end 913, no gap is maintained between the conventional biasing member 988 and the plunger 700, 700a. In fact, a portion of the buckled conventional biasing member 988, for example, at point 951 contacts one of the arms 788a of the plunger. Because the projections 790a and 790b have not completely left the annular collar 905, they have not yet come in contact with the buckled conventional biasing member 988. At this point during the expansion of the conventional biasing member 988, the plunger 700, 700a is still driven distally along the centerline of the longitudinal axis 150.

FIG. 12 is a cross-sectional view of the plunger 700, 700a, gripping region 113, 213, and the helical biasing member 188 of FIG. 10 after the helical biasing member 188 has further expanded from its compressed state. As discussed above in connection with FIG. 10, once the projections 790a and 790b are disengaged from the radial stop 101 of the gripping region 113, 213, the helical biasing member 188 drives the plunger 700, 700a toward the distal end of the automatic injection device 100, 200 along the longitudinal axis 150. As can be seen in this embodiment, because the helical biasing member 188 has a barrel design with the inner diameter of the coils at its middle portion greater than the inner diameter of the coils at its terminal ends, the helical biasing member 188 does not buckle and maintains an annular gap 910 between itself and the projections 790a and 790b of the plunger 700, 700a.

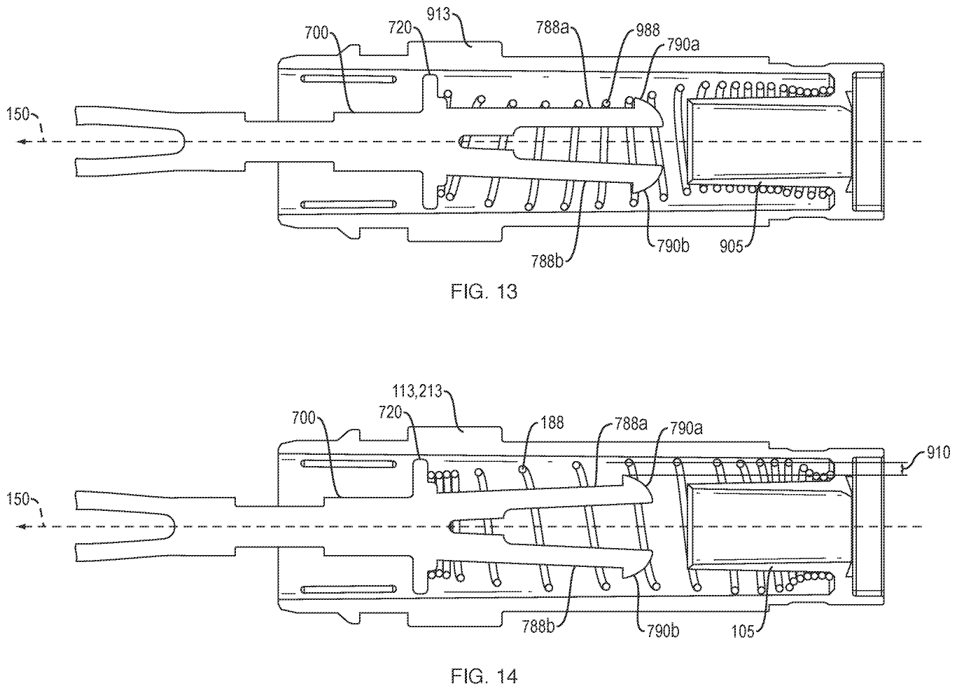

FIG. 13 is a cross-sectional view of the plunger 700, 700a, the gripping region 913, and the conventional biasing member 988 of FIG. 9 after the plunger arms 788a, 788b have moved past the distal end of the annular collar 905. After the conventional biasing member 988 has propelled the plunger 700, 700a toward the distal end of the automatic injection device along the longitudinal axis 150 past the distal end of the annular collar 905, the flexible legs 788a and 788b of the plunger 700, 700a expand outwardly toward the coils of the conventional biasing member 988, and the projections 790a and 790b snag onto a portion of the buckled conventional biasing member 988. This undesirable interaction between the projections 790a and 790b of the plunger 700, 700a and the conventional biasing member 988 causes a temporary loss of force on the syringe plunger, which can result in a wet injection event, as described above. This interaction also alters the trajectory of the plunger 700, 700a away from the center line of the longitudinal axis 150. This altered trajectory will be discussed in more detail in reference to FIG. 15.

FIG. 14 is a cross-sectional view of the plunger 700, 700a, the gripping region 113, 213, and the helical biasing member 188 of FIG. 10 as the plunger 700, 700a moves past the distal end of the annular collar 105, according to an exemplary embodiment. As can be seen in this embodiment, the plunger 700, 700a has been propelled toward the distal end of the automatic injection device 100, 200 along the center line of the longitudinal axis 150 to the point where the projections 790a and 790b of the plunger 700, 700a have exited the distal end of the annular collar 105. After exiting the annular collar 105, the flexible arms 788a and 788b expand outwardly toward the coils of the helical biasing member 188. However, because the helical biasing member 188 does not buckle, the annular gap 910 is maintained between the helical biasing member 188 and the projections 790a and 790b of the plunger 700, 700a. As illustrated in FIGS. 10, 12, and 14, the size of the annular gap 910 varies throughout the injection process. Nevertheless, a gap 910 is maintained throughout the entire expansion of the helical biasing member 188, such that there is no undesirable interactions between the plunger 700, 700a and the helical biasing member 188. Because the projections 790a and 790b of the plunger 700, 700a do not interact with or snag the coils of the helical biasing member 188, there is no temporary loss of power that is attributable to a wet injection event. As discussed above, this is achievable without increasing the amount of confined inner space within the automatic injection device 100, 200.

FIG. 15 is a cross-sectional view of the plunger 700, 700a, the gripping region 913, and the conventional biasing member 988 of FIG. 9 as the conventional biasing member 988 nears full expansion within the confined space of the automatic injection device. Once the conventional biasing member 988 has snagged against one of the protrusions 790a or 790b of the plunger 700, 700a, as shown in FIG. 13, the trajectory of the plunger 700, 700a is altered away from the longitudinal axis 150. In this example, the orientation of the plunger 700, 700a is offset by an angle θ, rather than continuing straight along the center line of the longitudinal axis 150.

Those skilled in the art will appreciate that the range of the offset angle θ is expected to be larger in an automatic injection device implemented with the straight plunger 700a, as shown in FIG. 5B, as compared to the split plunger 700 shown in FIG. 5A. This is because the sides of the compressible portion 780 of the plunger 700 of FIG. 5A are constrained by the inner surface of the syringe during injection. In contrast, the straight plunger 700a does not contact the inner surface of the syringe and has a wider range of offset angles relative to the centerline of the longitudinal axis 150.

This misalignment can generate increased friction during operation of the automatic injection device and, in combination with the temporary loss of power caused by the snagging of the projections 790a, 790b on the conventional biasing member 988, can contribute to the occurrence of a wet injection event.

FIG. 16 is a cross-sectional view of the plunger 700, the gripping region 113, 213, and the helical biasing member 188 of FIG. 10 as the helical biasing member 188 nears full expansion within the confined space of the automatic injection device 100, 200, according to an exemplary embodiment. As can be seen, the plunger 700 has been propelled toward the distal end of the automatic injection device 100, 200 in a straight trajectory along the center line of the longitudinal axis 150. Because the helical biasing member 188 does not buckle, the annular gap 910 is maintained between the helical biasing member 188 and the projections 790a and 790b of the plunger 700 during the entire injection process, and the trajectory of the plunger 700 is not altered by any undesirable interactions with the helical biasing member 188. As illustrated in FIGS. 10, 12, 14, and 16, the annular gap 910 is maintained throughout the entire expansion of the helical biasing member 118, and a consistent force on the plunger 700 is maintained. Preventing undesirable interactions between the plunger 700 and the helical biasing member 188, preventing a temporary loss of power on the plunger 700, and maintaining the aligned trajectory of the plunger 700 combine to help prevent wet injection events.

FIG. 17 is a perspective view of an embodiment of the gripping region 113, 213. In some embodiments, the gripping region 113, 213 includes an inner surface 107 that defines a confined inner space within the automatic injection device 100, 200. The gripping region 113, 213 includes an annular collar 105 extending distally. In some embodiments, a plurality of ribbed protrusions 1701a-1701f are spaced about the inner surface 107. The ribbed protrusions 1701a-1701f are spaced circumferentially about the inner surface 107 and extend radially inwardly toward the center line of the longitudinal axis 150 of the gripping region 113, 213. The ribbed protrusions 1701a-1701f reduce the amount of confined inner space within the gripping region 113, 213 adjacent to the helical biasing member 188 (not shown). Reducing the amount of confined inner space within the gripping region 113, 213 limits the amount of space available within the housing 112, 212 for the syringe, the syringe plunger 700, and the helical biasing member 188. The reduced amount of confined inner space provided by the plurality of ribbed protrusions 1701a-1701f can provide added support to the helical biasing member 188 and help reduce buckling of the helical biasing member 188, in some embodiments, without significantly increasing the weight of the gripping region 113, 213 or friction between the inner surface 107 and the helical biasing member 188. Thus, the ribbed protrusions 1701a-1701f can help prevent buckling of the helical biasing member 188 and reduce interactions between the helical biasing member 188 and the syringe plunger 700. In this embodiment, the gripping region 113, 213 includes six ribbed protrusions 1701a-1701f spaced about the inner surface 107. In some embodiments, the gripping region 113, 213 also includes one or more tabs 127, 227 that may snap-fit into corresponding openings on a distal housing component, as discussed above, to ensure alignment and coupling of the components. The gripping region 113, 213 can also include contours 128, 228, in some embodiments, to facilitate gripping of the automatic injection device 100, 200.

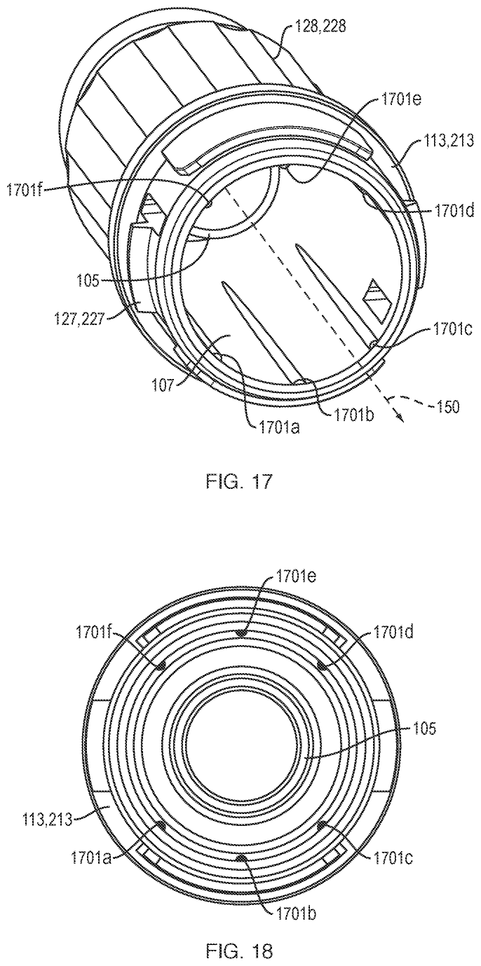

FIG. 18 is an end view of the embodiment of the proximal end 913 of FIG. 17, according to an exemplary embodiment. As can be seen, the ribbed protrusions 1701a-1701f are spaced about the inner surface of the proximal end 913 and extend radially inward, further limiting the amount of confined space between the housing 112, 212 and the annular collar 105.

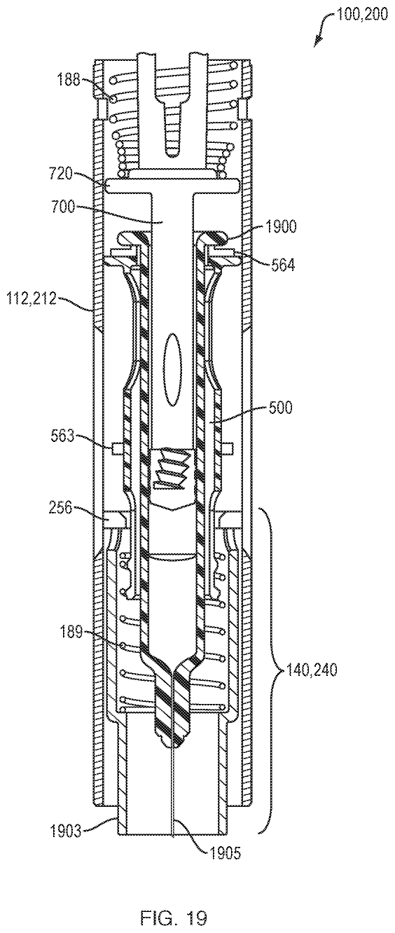

FIG. 19 illustrates a cross-sectional view of a portion of the automatic injection device 100, 200, according to an exemplary embodiment. As can be seen in this example, a syringe carrier 500 is configured to hold or contain at least a portion of a syringe 1900, which itself is contained within the housing 112, 212. The syringe 1900 may be configured to hold a medicament and may be manufactured using any suitable materials including glass, and polymer materials. The syringe 1900 may include one or more internal coatings or multiple layers with an oxygen or water barrier layer material, or both. In some examples, the syringe 1900 may be manufactured using co-extrusion or co-injection molding methods. In such examples in which co-extrusion or co-injection methods are used, the syringe 1900 may include a scratch-resistant layer, a barrier layer, and/or an inner low/non-leachable layer, such as one or more cyclo olefin polymer (COP) or cyclo olefin copolymer (COC) layers. The barrier layer may be disposed as a middle layer relative to an outer scratch-resistant layer and an inner non-leachable layer. The syringe 1900 may be configured to hold any suitable volume of medicament. In some examples, the syringe may hold a volume of about 0.05 to about 1.4 mL; in some examples, the syringe may hold a volume of about 1.5 to about 3.0 mL.

The needle 1905 of the syringe 1900 may have any suitable size, such as, for example, an inner diameter between about 0.15 to about 0.27 mm.

During operation, a syringe lockout shroud 1903 is depressed against the injection site by the user. In some embodiments, the syringe lockout shroud 1903 has a substantially tubular body through which the syringe needle 1905 can project during operation of the device. Upon activation of the automatic injection device 100, 200, the helical biasing member 188 pushes against the retaining flange 720 of the syringe plunger 700 and urges the syringe 1900 toward the distal end 140, 240 of the automatic injection device 100, 200. The housing 112, 212 is configured to limit the movement of the syringe carrier 500 beyond the distal end 140, 240 of the housing 112, 212, and the syringe carrier 500 in turn limits the movement of the syringe 1900. In some embodiments, the syringe carrier 500 includes one or more intermediate flanges 563 that can interact with an interior stop 256 on the housing 112, 212 to limit forward motion of the syringe carrier 500 and the syringe 1900. Once an injection has been completed, a secondary biasing member 189 drives the syringe carrier 500 along with the syringe 1900 toward the proximal end 141, 241 of the automatic injection device 100, 200.

In this particular embodiment, a damper 564 is positioned between a portion of the syringe 1900 and the syringe carrier 500. In some embodiments, the damper 564 is formed of an elastomeric material such as a high impact thermoplastic elastomer TPE and assists in absorbing force between the syringe carrier 500 and the syringe 1900 from the helical biasing member 188. The syringe 1900 may be formed of glass, in some embodiments, and the damper 564 helps prevent the syringe 1900 from breaking under the force of the helical biasing member 188. The damper 564 may have any suitable size and shape, for example, as shown in FIG. 19, the damper 564 may have a ring shape configured to increase contact of the damper 564 to the contact area of the flange of the syringe 1900 to reduce contact pressure. In some embodiments in addition or as an alternative to the damper 546 positioned between a portion of a proximal flange portion of the syringe 1900 and the syringe carrier, as shown in FIG. 19, the damper 564 may be positioned at other position(s) between the syringe 1900 and the syringe carrier 500. In some examples, the damper 564 may have a sleeve shape positioned at a distal portion of the syringe 1900 between the syringe 1900 and the syringe carrier.

Alternatively, plastic syringes can be used, instead of glass syringes, in order to reduce breakage occurrences of the syringe, which may result from an increase in the expansion force of the helical biasing member 188. However, solid plastic materials, such as cyclic olefin polymer (COP) or cyclic olefin copolymer (COC) materials, although they can be lighter and resist breakage more than glass, may provide the same oxygen and water moisture barrier properties. In some embodiments, a multi-layered polymer structure is used that can provide increased resistance to breakage as well as an oxygen and water moisture barrier similar to that of glass. An example of one such multi-layered structure includes one or more layers of a COP or COC resin, and an oxygen absorbing material, such as ethylene methyl acrylate cyclohexene methyl acrylate (EMCM) or poly EMCM. Such a multi-layer structure can mitigate syringe breakage and also provide an oxygen and water moisture barrier similar to glass. In some embodiment, such a multi-layer structure can be used in syringes or medicament containers for protein-based drug products. In some embodiments, such a multi-layer structure can facilitate storage of oxygen sensitive medicaments for up to two years.

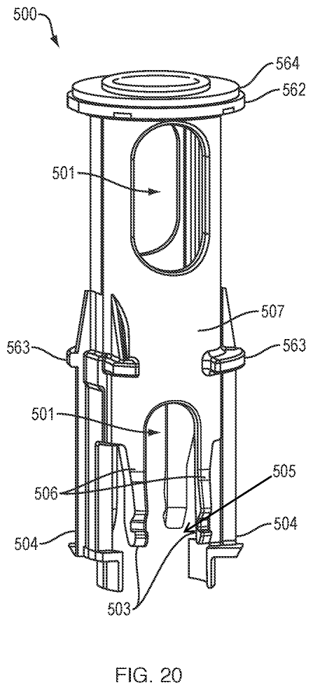

FIG. 20 illustrates an example syringe carrier 500, according to an exemplary embodiment. In this example embodiment, the syringe carrier 500 has a substantially tubular body including two distal openings 505 at the distal end of the syringe carrier 500. The distal openings 505 are on opposing sides of the syringe carrier 500, in this embodiment. The syringe carrier 500 also includes two proximal openings 501 on opposing sides of the syringe carrier 500. The distal openings 505 are defined, at least in part, by two pairs of legs 506 extending from a middle portion 507 of the syringe carrier 500. The middle portion 507 is disposed between the distal openings 505 and the proximal openings 501.

In some embodiments, the middle portion 507 is sized and configured to provide suitable strength to the syringe carrier 500 to prevent breaking or deformation during operation of the device. The distal openings 505 and the proximal openings 501 can be configured such that, once assembled within an automatic injection device, such as the automatic injection device shown in FIG. 2, they align with the elongated window 230 at various stages of operation of the automatic injection device. In some embodiments, each of the pairs of legs 506 can include an anchor portion 503 at a distal end of each leg. The anchor portion 503 can include one or more projections to define a generally radial groove and to engage an interior stop or feature within the housing of the automatic injection device.

The damper 564 can be fastened to or otherwise engage a flanged proximal end 562 of the syringe carrier 500. The syringe carrier 500 can also include a syringe carrier coupler 504, in some embodiments, formed as two beams extending from the middle portion 507 beyond the anchor portion 503 to facilitate coupling of the syringe carrier 500 with an end of a secondary biasing member, such as the second biasing member 189. The syringe carrier 500 can also include, in some embodiments, one or more intermediate flanges 563 that can interact with the interior stop 256, or flange, of the syringe housing 112, 212 of the automatic injection device 100, 200, as discussed above, to limit forward movement of the syringe carrier 500 and the syringe 1900. The intermediate flanges 563 of the syringe carrier 500 extend radially outward from the middle portion 507 and can halt the forward movement of the syringe carrier 500 before the syringe carrier coupler 504 comes in contact with the syringe lockout shroud 1903.

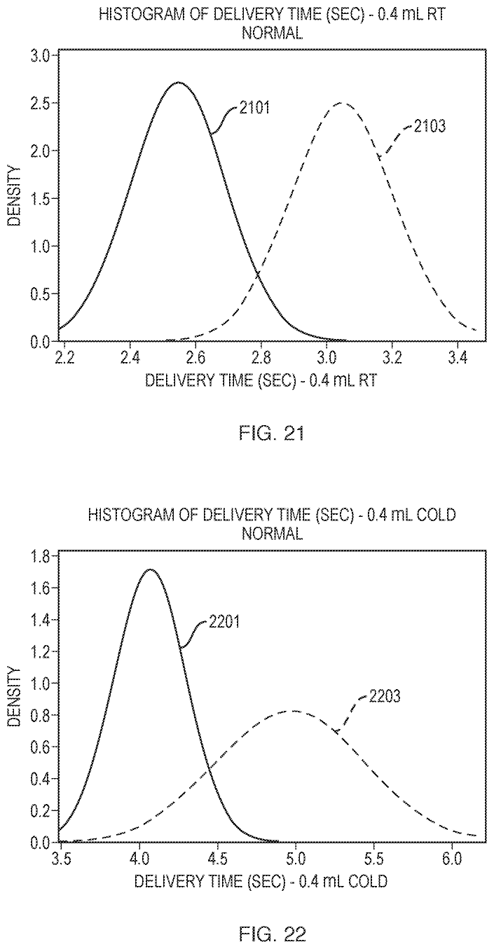

As discussed above, the barrel design of the helical biasing member 188 reduces or eliminates interactions between the helical biasing member 188 and the plunger 700, and results in a shorter delivery time than with a conventional biasing member 988. FIG. 21 is a graphical comparison of the delivery time, in seconds, of two different automatic injection devices for delivering 0.4 mL of a medicament at room temperature (about 23° C.), according to an exemplary embodiment.

As used herein, the “delivery time” refers to the amount of time it takes for an automatic injection device to substantially empty the contents of the reservoir through a 29 gauge needle of the device into air upon being activated.

The delivery time 2101 for the automatic injection device 100, 200 using the helical biasing member 188 disclosed herein was significantly less than the delivery time 2103 for a conventional automatic injection device of like design using a conventional biasing member 988. The example data used to generate the plots of FIG. 21 represent an average of thirty samples from each different automatic injection device, and the density on the y-axis represents the distribution of results from the thirty test samples. For the comparison shown in FIG. 21, helical biasing member 188b as taught herein was compared to a conventional biasing member 988. The helical biasing member 188b was noted to reduce the delivery time by approximately 0.5 seconds, as compared to the conventional biasing member 988, while at room temperature. The mean delivery time for the automatic injection device 100, 200 using the helical biasing member 188b as taught herein and plotted in FIG. 21 was about 2.544 seconds, with a standard deviation of about 0.1465. The mean delivery time for a conventional automatic injection device using the conventional biasing member 988 and plotted in FIG. 21 was about 3.047 seconds, with a standard deviation of about 0.1599. The shortened delivery time can be attributed to the lack of undesirable interactions between the plunger 700 and the helical biasing member 188b, as discussed above. It should be appreciated that while helical biasing member 188b was used for the comparison shown in FIG. 21, other exemplary embodiments of the helical biasing member 188, such as helical biasing members 188a and 188c-h, also cause reductions in the delivery time compared to the conventional biasing member 988 under the same delivery conditions.

FIG. 22 is a graphical comparison of the delivery time, in seconds, of two different automatic injection devices for delivering 0.4 mL of a medicament at a refrigerated temperature (between about 2-8° C.), according to an exemplary embodiment. The delivery time 2201 for the automatic injection device 100, 200 using the helical biasing member 188 disclosed herein was significantly less than the delivery time 2203 for a conventional automatic injection device of like design using a conventional biasing member 988. The example data used to generate the plots of FIG. 22 represent an average of thirty samples from each different automatic injection device, and the density on the y-axis represents the distribution of results from the thirty test samples. For the comparison shown in FIG. 22, helical biasing member 188b as taught herein was compared to the conventional biasing member 988. The helical biasing member 188b was noted to reduce the delivery time by approximately 1.0 seconds, as compared to the conventional biasing member 988, while refrigerated when dispensing the medicament. The mean delivery time for the automatic injection device 100, 200 using the helical biasing member 188b as taught herein and plotted in FIG. 22 was about 4.065 seconds, with a standard deviation of about 0.2320. The mean delivery time for a conventional automatic injection device using the conventional biasing member 988 and plotted in FIG. 22 was about 4.968 seconds, with a standard deviation of about 0.4835. The shortened delivery time can be attributed to the lack of undesirable interactions between the plunger 700 and the helical biasing member 188b, as discussed above. It should be appreciated that while the helical biasing member 188b was used for the comparison shown in FIG. 22, other exemplary embodiments of the helical biasing member 188, such as the helical biasing members 188a and 188c-h, also cause reductions in the delivery time compared to the conventional biasing member 988 under the same delivery conditions.

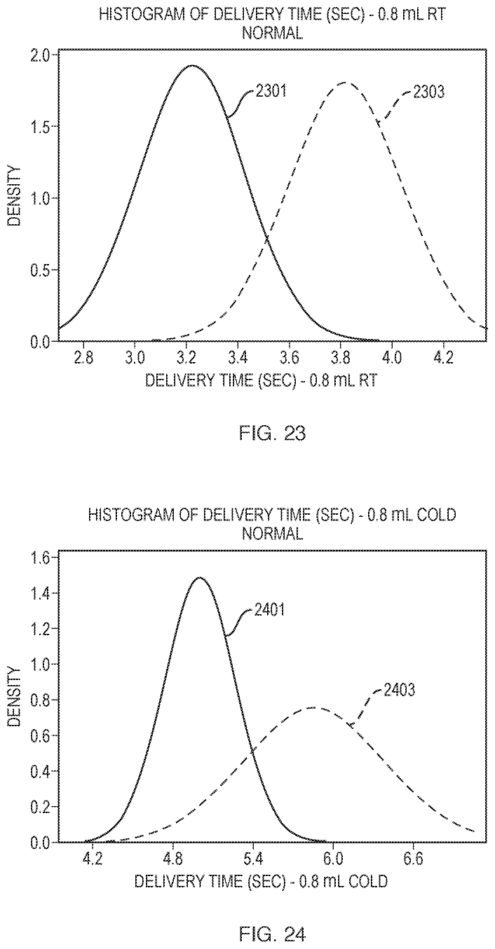

FIG. 23 is a graphical comparison of the delivery time, in seconds, of an automatic injection device for delivering 0.8 mL of a medicament at room temperature (about 23° C.), according to an exemplary embodiment. The delivery time 2301 for the automatic injection device 100, 200 using the helical biasing member 188 disclosed herein was significantly less than the delivery time 2303 for a conventional automatic injection device of like design using a conventional biasing member 988. The example data used to generate the plots of FIG. 23 represent an average of thirty samples from each different automatic injection device, and the density on the y-axis represents the distribution of results from the thirty test samples. For the comparison shown in FIG. 23, helical biasing member 188b as taught herein was compared to the conventional biasing member 988. The helical biasing member 188b was noted to reduce the delivery time by approximately 0.5 seconds, as compared to the conventional biasing member 988, while at room temperature. The mean delivery time for the automatic injection device 100, 200 using a helical biasing member 188b as described in FIG. 23 was about 3.221 seconds, with a standard deviation of about 0.2078. The mean delivery time for an automatic injection device using the conventional biasing member 988 and plotted in FIG. 23 was about 3.819 seconds, with a standard deviation of about 0.2217. The shortened delivery time can be attributed to the lack of undesirable interactions between the plunger 700 and the helical biasing member 188b, as discussed above. It should be appreciated that while helical biasing member 188b was used for the comparison shown in FIG. 23, other exemplary embodiments of the helical biasing member 188, such as helical biasing members 188a and 188c-h, also cause reductions in the delivery time compared to the conventional biasing member 988 under the same delivery conditions.

FIG. 24 is a graphical comparison of the delivery time, in seconds, of two different automatic injection devices for delivering 0.8 mL of a medicament at a refrigerated temperature (between about 2-8° C.), according to an exemplary embodiment. The delivery time 2401 for an automatic injection device 100, 200 using the helical biasing member 188 disclosed herein was significantly less than the delivery time 2403 for a conventional automatic injection device of like design using a conventional biasing member 988. The example data used to generate the plots of FIG. 24 represents an average of thirty samples from each different automatic injection device, and the density on the y-axis represents the distribution of results from the thirty test samples. For the comparison shown in FIG. 24, helical biasing member 188b as taught herein was compared to the conventional biasing member 988. The barrel design of the helical biasing member 188b was noted to reduce the delivery time by approximately 1.0 seconds, as compared to the conventional biasing member 988, while refrigerated when dispensing the medicament. The mean delivery time for an automatic injection device using the helical biasing member 188b as taught herein and plotted in FIG. 24 was about 5.0 seconds, with a standard deviation of about 0.2683. The mean delivery time for an automatic injection device using the conventional biasing member 988 and plotted in FIG. 24 was about 5.855 seconds, with a standard deviation of about 0.5307. The shortened delivery time can be attributed to the lack of undesirable interactions between the plunger 700 and the helical biasing member 188b, as discussed above. It should be appreciated that while helical biasing member 188b was used for the comparison shown in FIG. 24, other exemplary embodiments of the helical biasing member 188, such as helical biasing members 188a and 188c-h, also cause reductions in the delivery time compared to the conventional biasing member 988 under the same delivery conditions.

An experiment was conducted comparing the dispensing time for an automatic injection device using the helical biasing member 188, as disclosed herein, and a conventional automatic injection device using a conventional biasing member. Table 1 shows a comparison of the delivery time for the automatic injection device, as taught herein, using an embodiment of the helical biasing member 188 and the conventional automatic injection device using the conventional biasing member at room temperature, according to an exemplary embodiment. In this experiment, thirty automatic injection devices, as taught herein, were tested using various embodiments of the helical biasing member 188, as disclosed herein, and twenty conventional automatic injection devices were tested using a conventional biasing member. All the automatic injection devices were configured to deliver 0.8 mL of a solution.

TABLE 1

| | Medicament Delivery | Medicament Delivery |
|---|---|---|
| Sample No. | Time (sec): RT | Time (sec): RT |
| Test at Room | (Helical Biasing Member) | (Conventional Biasing |
| Temp (RT) | N = 30 Pens | Member) N = 20 Pens |
| Mean: | 4.87 | 6.05 |
| StDev: | 0.35 | 0.23 |
| Min: | 4.13 | 5.75 |
| Max: | 5.49 | 6.66 |

As can be seen in Table 1 above, the helical biasing member 188 disclosed herein resulted in a reduced delivery time of about 1.0 seconds, as compared to the conventional biasing member when dispensing the same volume of the same medicament. In this particular experiment, the automatic injection devices were tested at room temperature dispensing 0.8 mL of the same medicament from a pre-filled syringe.

As shown in Table 2, the delivery times for the helical biasing member was below 10 seconds at cold temperatures.

TABLE 2

| | Delivery Time in Air | | Delivery Time in Air | |
|---|---|---|---|---|
| | 0.8 mL Autoinjector | | 0.4 mL Autoinjector | |
| | with Conventional | | with Helical | |
| | Biasing Member | | Biasing Member | |
| | Room | | Room | |
| | Temp. | Cold | Temp. | Cold |
| Mean | 5.6 | 7.5 | 3.2 | 4.2 |
| SD | 0.6 | 0.7 | 0.3 | 0.4 |
| Min | 4.6 | 6.2 | 2.7 | 3.2 |
| Max | 7.0 | 9.4 | 3.9 | 5.5 |

Another experiment was conducted comparing delivery times of a conventional automatic injection device using a conventional biasing member to deliver 0.8 mL of a medicament against delivery times of an automatic injection device, as taught herein, using the helical biasing member 188 to deliver 0.4 mL of the same medicament. Table 2 above shows a comparison of delivery times between an automatic injection device configured to deliver 0.8 mL of a medicament at room temperature (about 23° C.) and cold temperatures (between about 2-8° C.) using the conventional biasing member. Table 2 also shows a comparison of delivery times between an automatic injection device, as taught herein, configured to deliver 0.4 mL of a medicament at room temperature and cold temperatures using an embodiment of the helical biasing member 188. In this particular experiment, the automatic injection devices were tested dispensing the same medicament but at different volumes from a pre-filled syringe.

Those skilled in the art will appreciate that at colder temperatures, the medicament being delivered increases in viscosity, thus resulting in an increased delivery time compared to delivery at room temperature. Once the automatic injection device is removed from a refrigerated environment, the medicament slowly increases in temperature, thus decreasing in viscosity and resulting in a decreased delivery time as the medicament warms. Various types of medicaments are often stored at cold temperatures in a refrigerator. In such scenarios, if a user removes the automatic injection device from a refrigerator and immediately performs an injection, the viscosity of the medicament and the delivery time of the automatic injection device will be greater than if the user allows the medicament to warm outside of the refrigerated environment before performing the injection. The use of a helical biasing member 188, as disclosed herein, provides a significant reduction in delivery time at both cold and room temperatures.

TABLE 3

| | Delivery Time in Air | | Delivery Time in Air | |
|---|---|---|---|---|
| | 0.8 mL Autoinjector | | 0.8 mL Autoinjector | |
| | with Conventional | | with Helical | |
| | Biasing Member | | Biasing Member | |
| | Room | | Room | |
| | Temp. | Cold | Temp. | Cold |
| Mean | 5.6 | 7.5 | 4.6 | 6.1 |
| SD | 0.6 | 0.7 | 0.5 | 1.0 |
| Min | 4.6 | 6.2 | 3.9 | 4.3 |
| Max | 7.0 | 9.4 | 6.1 | 8.5 |

Another experiment was conducted comparing delivery times of a conventional automatic injection device using a conventional biasing member to deliver 0.8 mL of a medicament against delivery times of an automatic injection device, as taught herein, using an embodiment of the helical biasing member 188 to deliver 0.8 mL of a medicament. Table 3 above shows a comparison of delivery times between a conventional automatic injection device configured to deliver 0.8 mL of the same medicament at room temperature (about 23° C.) and cold temperatures (between about 2-8° C.) using the conventional biasing member. Table 3 also shows a comparison of delivery times between an automatic injection device configured to delivery 0.8 mL of the same medicament at room temperature and at cold temperatures using an embodiment of the helical biasing member 188. As can be seen in Table 3, the use of the helical biasing member 188 noticeably reduces delivery time, as compared to the use of a conventional biasing member for the same medicament.

In one example embodiment, an automatic injection device is implemented with the helical biasing member 188, as described herein, having an expansion force between the range of about 10N to about 50N. The automatic injection device can deliver about 1.0 mL of a medicament having a viscosity in the range of about 1.0 to about 30.0 mPa·s with a delivery time equal to or less than 15 seconds after approximately thirty minutes of warm-up time in room temperature from a refrigerated storage environment of between about 2.0 to about 8.0 degrees Celsius. In some examples the automatic injection device can deliver about 1.0 mL of a medicament having a viscosity in the range of about 1.0 to about 30.0 mPa·s with a delivery time equal to or less than 10 seconds after approximately thirty minutes of warm-up time in room temperature from a refrigerated storage environment of between about 2.0 to about 8.0 degrees Celsius.

In exemplary embodiments, the expansion length of the helical biasing member 188 during injection of the medicament can be determined for an automatic injection device. For example, the automatic injection device can begin delivering the medicament through the needle of the syringe when the expansion length of the helical biasing member is about 39 mm, and the delivery of the medicament can be completed when the expansion length of the helical biasing member is about 70 mm Often, the expansion force of the helical biasing member is greater when the helical biasing member is in a compressed state and the expansion length is shorter. A number of helical biasing members having maximum expansion forces between about 25N to about 70N were tested and their expansion forces were measured as their expansion lengths increased from a compressed state to an expanded state. It has been discovered that a more consistent expansion force during delivery of the medicament, when the expansion length of the example helical biasing member is between 39 mm and 70 mm, reduces the likelihood of breakage in the syringe. It has also been discovered that a helical biasing member with a lower maximum expansion force value of around 25N exerted a more consistent expansion force during delivery of the medicament when the expansion length of is between 39 mm and 70 mm Thus, a helical biasing member configured with a lower initial expansion force and a slower decay in force during expansion can reduce syringe breakage.

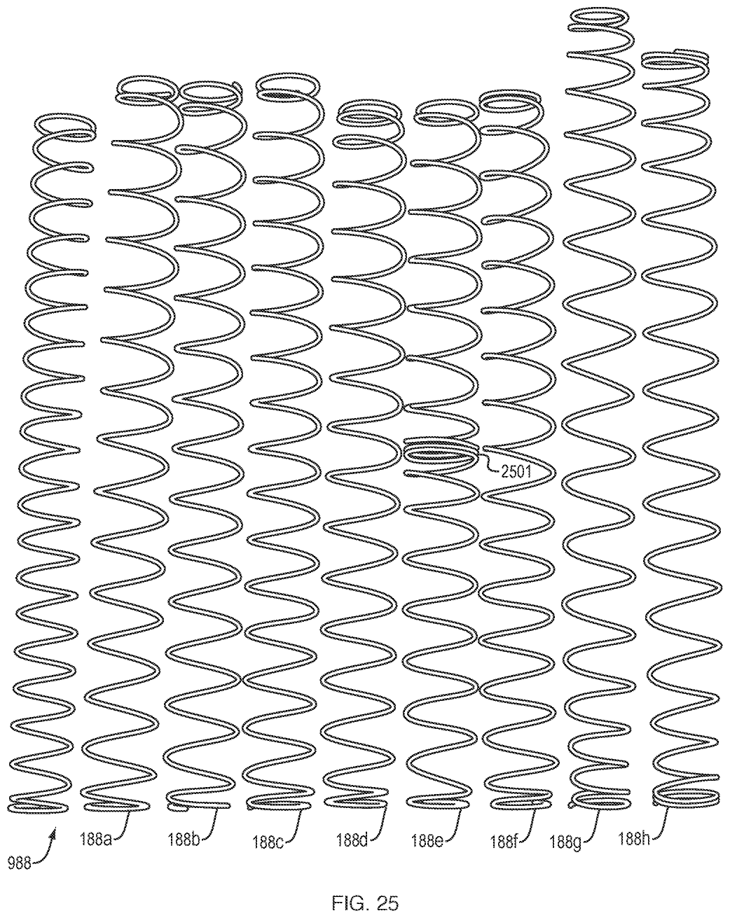

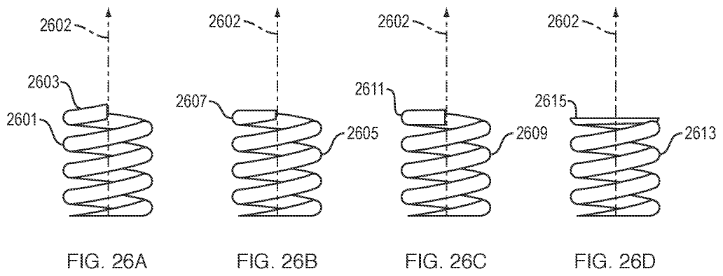

FIG. 25 illustrates the conventional biasing member 988 and eight different embodiments of the helical biasing member 188 with a barrel design, as taught herein. The conventional biasing member 988 is shown with a constant inner diameter along its entire length. The helical biasing members 188a-188h each have a larger inner diameter at their middle portions, than at the terminal ends. The various embodiments of the helical biasing members 188a-188h can be made of different materials, in some embodiments, and can have different lengths and numbers of coils. In one exemplary embodiment, the helical biasing member 188e includes two dead coils 2501 in its middle portion that provide no biasing force to the helical biasing member 188e and which are in contact with an adjacent coil. FIG. 26A illustrates an example embodiment for the terminal end 2601 of the helical biasing member 188, as disclosed herein. In some embodiments, the helical biasing member 188 can include a dead end coil 2603 at the terminal end 2601 that does not contribute to the expansion force of the helical biasing member 188. In this particular embodiment, the dead end coil 2603 is a plain end coil that retains its angle of inclination with respect to a central axis 2602. Those skilled in the art will recognize that the terminal end 2601 has an open and not-ground end design.

FIG. 26B illustrates another example embodiment for the terminal end 2605 of the helical biasing member 188, as disclosed herein. In some embodiments, the helical biasing member 188 can include a dead end coil 2607 at the terminal end 2605 that does not contribute to the expansion force of the helical biasing member 188. In this particular embodiment, the dead end coil 2607 is ground to a flat surface perpendicular to the central axis 2602. Those skilled in the art will recognize that the terminal end 2605 has an open and ground end design.

FIG. 26C illustrates another example embodiment for the terminal end 2609 of the helical biasing member 188, as disclosed herein. In some embodiments, the helical biasing member 188 can include a dead end coil 2611 at the terminal end 2609 that does not contribute to the expansion force of the helical biasing member 188. In this particular embodiment, the dead end coil 2611 is squared or bent perpendicular to the central axis 2602 such that it no longer retains the angle of inclination of the other coils. Those skilled in the art will recognize that the terminal end 2609 has a closed and not-ground end design.

FIG. 26D illustrates another example embodiment for the terminal end 2613 of the helical biasing member 188, as disclosed herein. In some embodiments, the helical biasing member 188 can include a dead end coil 2615 at the terminal end 2613 that does not contribute to the expansion force of the helical biasing member 188. In this particular embodiment, the dead end coil 2615 is squared, bent perpendicular to the central axis 2602 of the helical biasing member 188, and is also ground to a flat surface perpendicular to the central axis 2602. Those skilled in the art will recognize that the terminal end 2613 has a closed and ground end design.

In addition to factors discussed above, other factors can contribute to a wet injection event. For example, in a conventional automatic injection device, a portion of the expansion force of the conventional biasing member 988 can be transferred from the syringe carrier to the skin of the user at the injection site through the syringe lockout shroud. This transfer of force is the result of a physical interaction between a portion of the syringe carrier and the syringe lockout shroud, which is held against the skin of the user at the injection site. This transfer of force can provoke a reaction in the user to prematurely remove the conventional automatic injection device away from the injection site before the injection is complete.

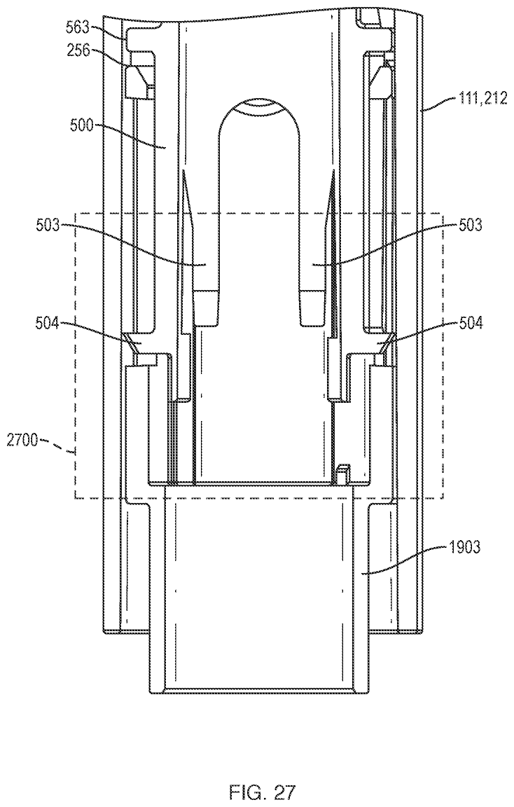

FIG. 27 illustrates a solution to the problem of transferring the expansion force of the helical biasing member 188 to the injection site via the syringe lockout shroud 1903. As illustrated in FIG. 27, a portion of the expansion force of the helical biasing member 188 is transferred to the housing 112, 212, which is not in contact with the injection site.

FIG. 27 illustrates a cross-sectional view of a portion of the automatic injection device 100, 200, as disclosed herein. In this example embodiment, the syringe lockout shroud 1903 is disposed within the housing 111, 212 of the automatic injection device 100, 200. When a user is prepared to operate the injection device, the syringe lockout shroud 1903 is depressed against the injection site. Once depressed against the injection site, the user activates the automatic injection device 100, 200 to cause the helical biasing member 188 to urge the syringe plunger 700, 700a and the syringe carrier 500 toward the distal end of the automatic injection device 100, 200. In this particular embodiment, the syringe carrier 500 includes anchor portions 503, syringe carrier couplers 504, and one or more intermediate flanges 563 extending radially outward. The anchor portions 503 can include one or more projections to define a generally radial groove and to engage a feature within the housing of the automatic injection device. The syringe carrier couplers 504 can be formed as two beams extending beyond the anchor portions 503 to facilitate coupling of the syringe carrier 500 with an end of a secondary biasing member, in some embodiments. During operation of the automatic injection device 100, 200, the intermediate flanges 563 interact with one or more of the interior stops 256 that extend radially inward from the housing 112, 212 to limit forward movement of the syringe carrier 500 and, in turn, the syringe 1900. In some embodiments, the interaction between the interior stops 256 (syringe housing flange) and the intermediate flanges 563 of the syringe carrier 500 halts the forward movement of the syringe carrier 500 before the syringe carrier coupler 504 comes in contact with the syringe lockout shroud 1903, thus preventing a transfer of force from the syringe carrier 500 to the syringe lockout shroud 1903 in region 2700.

Any transfer of force to the syringe lockout shroud 1903 from the syringe carrier 500 is undesirable because it may be transferred to the skin of the user at the injection site and cause the user to prematurely pull the automatic injection device 100, 200 away from the injection site to cause a wet injection event. Any force transferred to the injection site through the syringe lockout shroud 1903 can also make it more difficult for the user to firmly hold the automatic injection device 100, 200 in place during an injection. However, the interaction between the intermediate flanges 563 of the syringe carrier 500 and the interior stops 256 (syringe housing flange) of the housing 112, 212 directs the force of the syringe carrier 500 to the housing 112, 212, which does not contact the skin of the user at the injection site. The syringe lockout shroud 1903 contacts the skin of the user at the injection site.

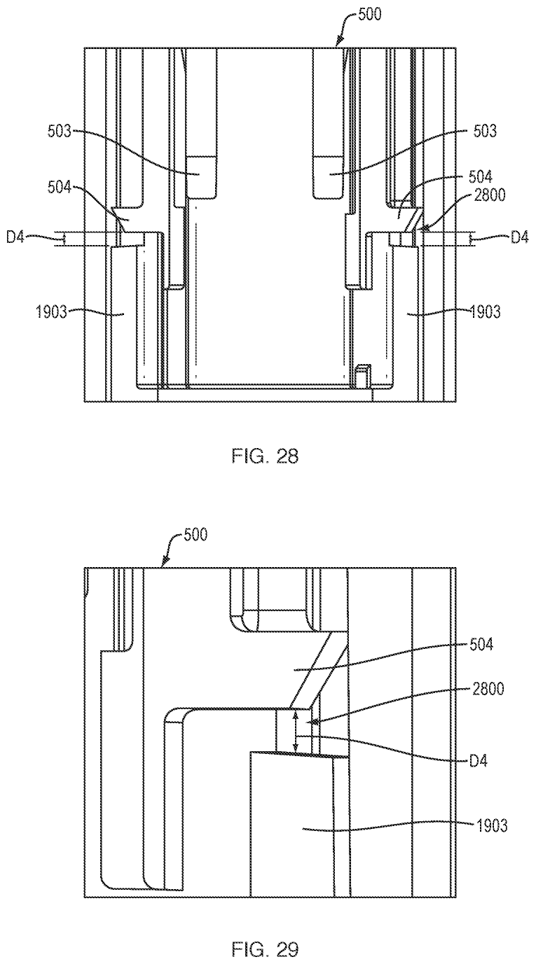

FIG. 28 illustrates an enlarged view of the region 2700 of FIG. 27 showing a portion of the syringe carrier 500 and the syringe lockout shroud 1903 for use in the automatic injection device 100, 200, as disclosed herein. In this example embodiment, the syringe carrier 500 includes the anchor portions 503 and the two syringe carrier couplers 504. As discussed above in reference to FIG. 27, the intermediate flanges 563 of the syringe carrier 500 halt the forward movement of the syringe carrier 500 before the syringe carrier coupler 504 can contact or interact with any interior portions of the syringe lockout shroud 1903. This allows a gap 2800 between the syringe carrier coupler 504 and the syringe lockout shroud 1903 having a length D4. In some embodiments, the gap length D4 is between about 0.70 mm to about 0.80 mm. This gap can be achieved without shortening the length of the syringe carrier, in some embodiments.

FIG. 29 illustrates another enlarged view of the region 2700 of FIG. 27 showing a portion of the syringe carrier 500 and syringe lockout shroud 1903. FIG. 29 illustrates a magnified view of the gap 2800, having a distance D4, between the syringe carrier coupler 504 and the syringe lockout shroud 1903. The gap 2800 is a result of the interaction between the intermediate flanges 563 of the syringe carrier 500 and the interior stops 256 of the housing 112, 212 that prevents contact between the syringe carrier coupler 504 and the syringe lockout shroud 1903.

Table 4 below shows a listing of the various embodiments of helical biasing members shown in FIG. 25. In some embodiments, the outer diameter of the helical biasing member has an average size of between about 12.45 mm and about 13.12 mm. In some embodiments, the expansion force of the helical biasing member is between about 10 N to about 66 N, for example, between 16.8 N and about 24.0 N. In some embodiments, the expansion force of the helical biasing member is between about 10 N to about 70 N. In some embodiments, the helical biasing member can define a spring rate constant k between 0.2 and 0.35, depending on the desired force of the helical biasing member. The inner diameter of the coils at each terminal end of the helical biasing member can be, for example, between about 9.0 mm and about 9.5 mm, in some embodiments. The inner diameter of the coils at the middle portion of the helical biasing member can be, for example, between about 10.45 mm and about 11.7 mm, in some embodiments. In some embodiments, the wire diameter (WD) of the helical biasing member is between about 0.75 mm and 1.0 mm, with the inner diameter (ID) of the helical biasing member at various points corresponding to the respective outer diameter (OD) of the helical biasing member minus the wire diameter multiplied by 2, i.e., ID=OD−2*WD. In some embodiments, the pitch of the coils of the helical biasing member can be between about 7.1 mm and about 7.8 mm, and the pitch angle can be between about 10.3 degrees and about 11.3 degrees. In some embodiments, the outer diameter of the coils at the middle portion of the helical biasing member is about 12.45 mm, and the diameter gradually tapers down to about 11.00 mm at each terminal end of the helical biasing member. In some embodiments, the outer diameter of the coils at the middle portion of the helical biasing member is about 12.50 mm, and the diameter gradually tapers down to about 10.8 mm at each terminal end of the helical biasing member. In one example embodiment, the tapering of the diameter of the coils begins about 1.5 to about 2.0 mm from each terminal end of the helical biasing member.

TABLE 4

| Helical Biasing | | Outer Dia. (mm) | Length (mm) | Number |
|---|---|---|---|---|
| Member Type | Material | Avg. ± Std. Dev. | Avg. ± Std. Dev. | of Coils |
| Type 1 | Music Wire- | 13.06 ± 0.11 | 127.3 ± 0.11 | 16 |
| (188a) | Galvanized | | | |
| Type 2 | Music Wire- | 13.00 ± 0.08 | 125.47 ± 0.40 | 16 |
| (188b) | Galvanized | | | |
| Type 3 | Music Wire- | 12.81 ± 0.03 | 126.06 ± 0.81 | 17 |
| (188c) | Galvanized | | | |
| Type 4 | 17-7 Stainless | 13.08 ± 0.07 | 122.48 ± 0.40 | 16 |
| (188d) | Steel | | | |
| Type 5 | 17-7 Stainless | 13.11 ± 0.03 | 121.45 ± 0.27 | 18 |
| (188e) | Steel-two | | | |
| | dead coils in | | | |
| | the middle | | | |
| Type 6 | 302 Stainless | 13.12 ± 0.02 | 123.57 ± 0.39 | 17 |
| (188f) | Steel | | | |
| Type 7 | EN 10270-3- | 12.45 ± 0.20 | 143 ± 4.2 | 18.5 |
| (188g) | 1.4310-HS | | | |
| | Stainless Steel | | | |
| Type 8 | EN 10270 Pt1 | 12.50 ± 0.16 | 131.00 ± 5.00 | 19 |
| (188h) | Patented | | | |
| | Carbon Steel | | | |

In another example embodiment, the inner diameter of the coils at each terminal end of the helical biasing member can be between about 8.5 mm and about 9.5 mm; and the outer diameter of the coils at the middle portion of the helical biasing member can be between about 12.45 mm and about 13.1 mm.

Table 5 below shows results of a sound test of automatic injection devices, as taught herein, using various embodiments of the helical biasing member 188, compared to a conventional automatic injection device using a conventional biasing member 988. Spring types FM-11 through FM-16, shown in Table 5, correspond to helical biasing members 188a-188f, respectively, described in reference to FIG. 25 and Table 4 above. The control biasing member corresponds to the conventional biasing member 988, described above. As can be seen in Table 5, embodiments of the helical biasing members 188a-188f did reduce coil-plunger interactions. This was determined based on a clicking sound caused by the plunger snagging a portion of the coils of a biasing member. With each embodiment of the helical biasing members 188a-188f, there was either no audible click detected, or the sound was softer than in the conventional automatic injection device of like design using the conventional biasing member 988.

TABLE 5

| | Testing Criteria | | | | | | | | | | |
|---|---|---|---|---|---|---|---|---|---|---|---|
| | Incomplete | | | | Delayed | | Glass | | Wet | | |
| Spring | Injection | | Clicking | | Delivery | | Breakage | | Injection | | |
| Type | Ctrl | Rib | Ctrl | Rib | Ctrl | Rib | Ctrl | Rib | Ctrl | Rib | Click Noise |
| FM-11 | No | No | No | No | No | No | No | No | No | No | N/A |
| FM-12 | No | No | No | 1 Pen | No | No | No | No | No | No | N/A |
| FM-13 | No | No | 6 Pens | 6 Pens | No | No | No | No | No | No | All Soft |
| FM-14 | No | No | 1 Pen | No | No | No | No | No | No | No | All Soft |
| FM-15 | No | No | No | No | No | No | No | No | No | No | All Soft |
| FM-16 | No | No | No | No | No | No | No | No | No | No | N/A |
| Control | No | No | 21 Pens | 20 Pens | No | No | No | No | No | No | Ctrl: 9 Loud, |
| | | | | | | | | | | | 12 Soft |
| | | | | | | | | | | | Rib: 11 Loud, |
| | | | | | | | | | | | 9 Soft |

Another experiment was conducted to determine delivery times of an automatic injection device, as taught herein, using an embodiment of the helical biasing member 188g (described in Table 4) to deliver 0.8 mL of a medicament into air. Table 6 below shows delivery times, calculated from 60 trials, of an automatic injection device configured to deliver 0.8 mL of the medicament at room temperature and at cold temperatures into air using an embodiment of the helical biasing member 188g. As can be seen in comparing the delivery time of the automatic injection device with helical biasing member 188g shown in Table 6 to, for example, the embodiment of the automatic injection device with a conventional biasing member shown in Table 3 to deliver the same volume of the same medicament at the same temperature into air, the use of the helical biasing member 188g noticeably reduces delivery time of the medicament, as compared to the use of a conventional biasing member for the same medicament under the same conditions.

TABLE 6

| | Delivery Time in Air | |
|---|---|---|
| | 0.8 mL Autoinjector | |
| | with Helical Biasing | |
| | Member | |
| | Room | |
| | Temp. | Cold |
| Mean | 3.69 | 4.76 |
| SD | 0.31 | 0.31 |
| Min | 2.94 | 4.13 |
| Max | 4.50 | 5.57 |

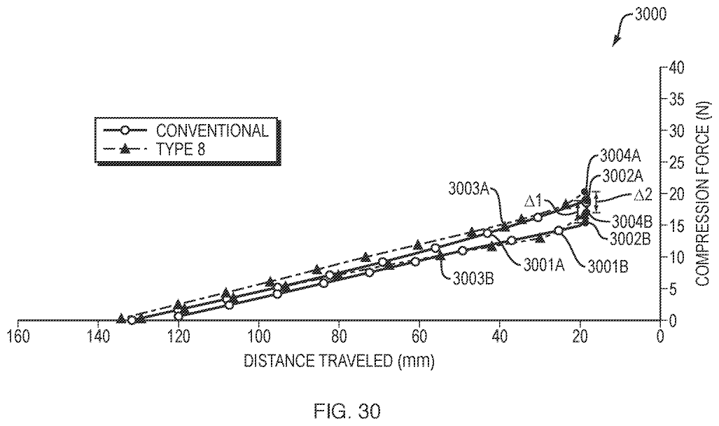

Referring now to FIG. 30, a graph 3000 illustrating force profiles of helical biasing member compression force vs. distance traveled (in mm) is presented for various helical biasing members. As can be seen, a conventional helical biasing member, such as helical biasing member 988, can have a compression force profile 3001A that linearly increases at one rate as the helical biasing member 988 is compressed to a maximum compression force point 3002. Upon the helical biasing member 988 being released from compression at the maximum compression force point 3002A, the helical biasing member 988 can have an extension force profile 3001B that linearly decreases at one rate as the helical biasing member 988 extends back to a relaxed position. A compression force drop A1 is observed when the helical biasing member 988 is released from compression at the maximum compression force point 3002A to an initial release compression force point 3002B, even though little appreciable extension of the helical biasing member 988 is observed, and is generally attributable to behavior of the spring 988 in a tightly constrained space.

An exemplary embodiment of a helical biasing member formed in accordance with the present invention, such as helical biasing member 188h, can have a compression force profile 3003A that linearly increases at two different rates as the helical biasing member 188h is compressed to a maximum compression force point 3004. Upon the helical biasing member 188h being released from compression at the maximum compression force point 3004A, the helical biasing member 188h can have an extension force profile 3003B which linearly decreases at the two different rates as the helical biasing member 188h extends back to a relaxed position. A compression force drop A2 is also observed when the helical biasing member 188h is released from compression at the maximum compression force point 3004A to an initial release compression force point 3004B, even though little appreciable extension of the helical biasing member 188h is observed, and is generally attributable to behavior of the spring 188h in a tightly constrained space.

As can be appreciated from FIG. 30, the force profiles 3001A, 3001B of the conventional helical biasing member 988 and the force profiles 3003A, 3003B of the exemplary embodiment of a helical biasing member 188h formed in accordance with the present invention can be similar. Thus, it should be appreciated that exemplary embodiments of helical biasing members 188 formed in accordance with the present invention can display similar spring behavior to conventional springs while contributing to the solution of addressing the undesirable effect of wet injection.

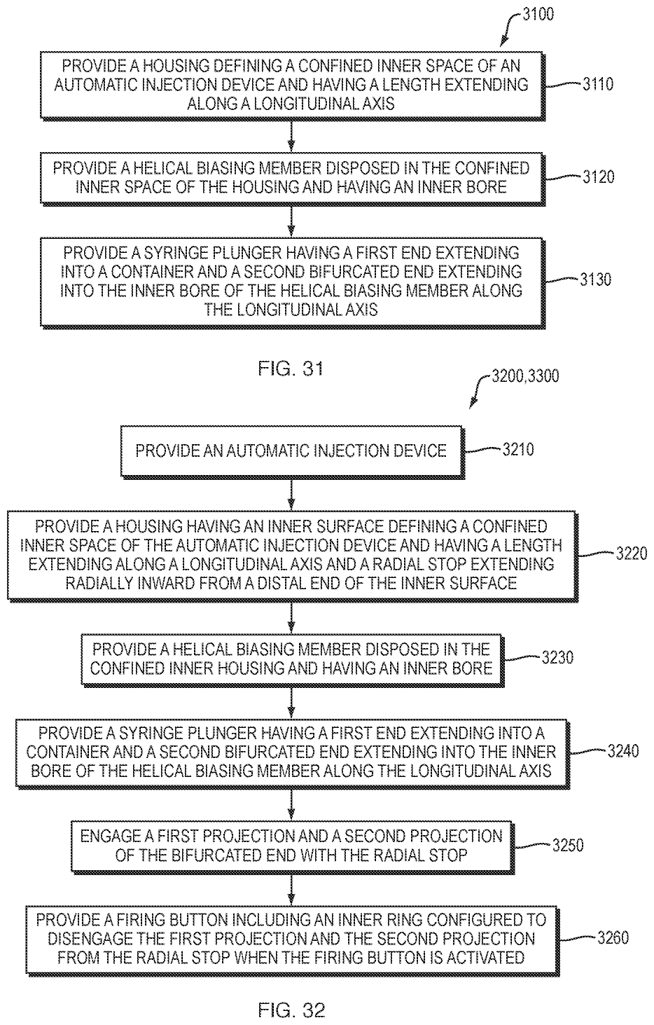

Referring now to FIG. 31, an exemplary embodiment of a method 3100 of forming an automatic injection device, such as automatic injection devices 100, 200, having a distal end 140 configured to deliver a medicament held in a container therein and a proximal end 141 configured to be controllable by a user is illustrated. The method 3100 includes providing 3110 a housing 112 defining a confined inner space of the automatic injection device 100, 200 and having a length extending along a longitudinal axis 150, such as from the proximal end 141 to the distal end 140. The method 3100 further includes providing 3120 a helical biasing member 188, which may be any of helical biasing members 188a-188h, disposed in the confined inner space of the housing 112. The helical biasing member 188 has an inner bore 800 and a length extending from a first terminal end 601 to a second terminal end 603. An inner diameter of the helical biasing member 188 at a middle portion 605 between the first terminal end 601 and the second terminal end 603 has a first inner diameter D3 greater than a second inner diameter D1, D2 at the first terminal end 601 or at the second terminal end 603, respectively. The method 3100 further includes providing 3130 a syringe plunger 700 having a first end extending into the container and a second bifurcated end extending into the inner bore 800 of the helical biasing member 188 along the longitudinal axis 150. The bifurcated end includes a first flexible arm 788a and a second flexible arm 788b, the first arm 788a having a first projection 790a at a first end thereof and the second arm 788b having a second projection 790b at a first end thereof, the first and second flexible arms 788a, 788b able to flex inwardly and outwardly relative to the longitudinal axis 150 within the inner bore 800 of the helical biasing member 188 while maintaining an annular gap 910 between the syringe plunger 700 and the helical biasing member 188. The helical biasing member 188 provided according to the method 3100 may, in some embodiments, be any of the previously described helical biasing members 188a-188h.

Referring now to FIG. 32, other exemplary embodiments of methods 3200, 3300 of forming an automatic injection device are illustrated. The method 3200 of forming an automatic injection device to reduce the occurrence of a wet injection includes providing 3210 an automatic injection device, such as automatic injection device 100, 200, having a distal end 140 configured to deliver a medicament held in a container therein and a proximal end 141 configured to be controllable by a user. The method 3200 further includes providing 3220 a housing 112 having an inner surface 107 defining a confined inner space of the automatic injection device 100, 200, the housing 112 having a length extending along a longitudinal axis 150, such as from the proximal end 141 to the distal end 140, and a radial stop 901 extending radially inwardly from a distal end of the inner surface 107. The method 3200 further includes providing 3230 a helical biasing member 188, which may be any of helical biasing members 188a-188h, disposed in the confined inner space of the housing 112. The helical biasing member 188 has an inner bore 800 and a length extending from a first terminal end 601 to a second terminal end 603. An inner diameter of the helical biasing member 188 at a middle portion 605 between the first terminal end 601 and the second terminal end 603 has a first inner diameter D3 greater than a second inner diameter D1, D2 at the first terminal end 601 or at the second terminal end 603, respectively. The method 3200 further includes providing 3240 a syringe plunger 700 having a first end extending into the container and a second bifurcated end extending into the inner bore 800 of the helical biasing member 188 along the longitudinal axis 150. The bifurcated end includes a first flexible arm 788a and a second flexible arm 788b, the first arm 788a having a first projection 790a at a first end thereof and the second arm 788b having a second projection 790b at a first end thereof, the first and second flexible arms 788a, 788b able to flex inwardly and outwardly relative to the longitudinal axis 150 within the inner bore 800 of the helical biasing member 188 while maintaining an annular gap 910 between the syringe plunger 700 and the helical biasing member 188. The first projection 790a and the second projection 790b engage 3250 the radial stop 901 to maintain the syringe plunger 700 in a latched position. The method 3200 further includes providing 3260 a firing button 132 including an inner ring configured to disengage the first projection 790a and the second projection 790b from the radial stop 901 when the firing button 132 by the user.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step to may be replaced with a plurality of elements or steps that serve the same purpose. Further, where parameters for various properties are specified herein for exemplary embodiments, those parameters may be adjusted up or down by 1/20th, 1/10^{th}, ⅕th, ⅓rd, ½nd, and the like, or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

What is claimed is:

1. An automatic injection device having a distal end configured to deliver a medicament held in a container therein and a proximal end configured to be controllable by a user, the automatic injection device comprising:
a housing defining a confined inner space of the automatic injection device, the housing having a length extending from the proximal end to the distal end along a longitudinal axis;
a helical biasing member disposed in the confined inner space of the housing along the longitudinal axis having an inner bore and a length extending from a first terminal end of the helical biasing member to a second terminal end of the helical biasing member opposite the first terminal end, an inner diameter of the helical biasing member at a middle portion between the first terminal end and the second terminal end having a first inner diameter greater than a second inner diameter at the first terminal end or at the second terminal end; and
a syringe plunger having a first end extending into the container and a second bifurcated end extending into the inner bore of the helical biasing member along the longitudinal axis, the bifurcated end having a first flexible arm and a second flexible arm, the first arm having a first projection at a first end thereof and the second arm having a second projection at a first end thereof, the first and the second flexible arms able to flex inwardly and outwardly relative to the longitudinal axis within the inner bore of the helical biasing member while maintaining an annular gap between the syringe plunger and the helical biasing member.

2. The automatic injection device of claim 1, wherein the first inner diameter of the helical biasing member at the middle portion is sized to prevent buckling of the helical biasing member during compression and expansion within the automatic injection device.

3. The automatic injection device of claim 1, wherein the second inner diameter of the helical biasing member is between 9.0 mm and 9.5 mm and the first inner diameter of the helical biasing member is between 11.4 mm and 11.7 mm.

4. The automatic injection device of claim 1, wherein the second inner diameter of the helical biasing member is between 8.5 mm and 9.5 mm, and an outer diameter of the middle portion of the helical biasing member is between 12.6 mm and 13.0 mm.

5. The automatic injection device of claim 1, wherein the helical biasing member further comprises at least one dead coil located in the middle portion.

6. The automatic injection device of claim 1 further comprising a syringe carrier configured to hold a syringe and a high impact material positioned between the syringe carrier and the syringe, the high impact material configured to reduce contact pressure between the syringe carrier and the syringe.

7. The automatic injection device of claim 1, wherein the helical biasing member has an expansion force between about 10 N to about 40 N.

8. The automatic injection device of claim 1, wherein the syringe plunger further comprises a retaining flange disposed to engage the first terminal end of the helical biasing member.

9. The automatic injection device of claim 1, wherein the first inner diameter of the middle portion of the helical biasing member defines a portion of the annular gap to provide sufficient space between the first projection and the second projection when the first and second arms flex outwardly to allow the helical biasing member to bias the syringe plunger toward the distal end of the automatic injection device unobstructed by the first projection and the second projection.

10. The automatic injection device of claim 1, wherein the housing comprises a hollow tubular member having an inner surface extending along the longitudinal axis defining the confined inner space.

11. The automatic injection device of claim 10, wherein the housing further comprises a plurality of ribbed protrusions circumferentially spaced about the inner surface, each ribbed protrusion extending radially inwardly along the longitudinal axis to reduce the confined inner space adjacent to the helical biasing member.

12. The automatic injection device of claim 10, wherein the proximal end of the housing further comprises a radial stop extending radially inward from the inner surface and configured to engage with the first projection and the second projection.

13. The automatic injection device of claim 12, wherein the proximal end of the housing further comprises an annular collar extending toward the distal end along the longitudinal axis within the confined inner space from the radial stop.

14. The automatic injection device of claim 12, further comprising a firing button including an inner ring configured to disengage the first projection and the second projection from the radial stop when the firing button is activated by the user.

15. The automatic injection device of claim 1, wherein an outer diameter of the middle portion is between 12.45 mm and 12.5 mm and an outer diameter of at least one of the first terminal end and the second terminal end is between 10.8 mm and 11.0 mm.

16. The automatic injection device of claim 1, wherein the helical biasing member has an expansion force between about 10 N to about 70 N.

17. A method of forming an automatic injection device having a distal end configured to deliver a medicament held in a container therein and a proximal end configured to be controllable by a user, the method comprising:
providing a housing defining a confined inner space of the automatic injection device, the housing having a length extending from the proximal end to the distal end along a longitudinal axis;
providing a helical biasing member disposed in the confined inner space of the housing along the longitudinal axis having an inner bore and a length extending from a first terminal end of the helical biasing member to a second terminal end of the helical biasing member opposite the first terminal end, an inner diameter of the helical biasing member at a middle portion between the first terminal end and the second terminal end having a first inner diameter greater than a second inner diameter at the first terminal end or at the second terminal end; and
providing a syringe plunger having a first end extending into the container and a second bifurcated end extending into the inner bore of the helical biasing member along the longitudinal axis, the bifurcated end having a first flexible arm and a second flexible arm, the first arm having a first projection at a first end thereof and the second arm having a second projection at a first end thereof, the first and the second flexible arms able to flex inwardly and outwardly relative to the longitudinal axis within the inner bore of the helical biasing member while maintaining an annular gap between the syringe plunger and the helical biasing member.

18. The method of claim 17, wherein the first inner diameter of the helical biasing member at the middle portion is sized to prevent buckling of the helical biasing member during compression and expansion within the automatic injection device.

19. The method of claim 17, wherein the first inner diameter of the middle portion of the helical biasing member defines a portion of the annular gap to provide sufficient space between the first projection and the second projection when the first and second arms flex outwardly to allow the helical biasing member to bias the syringe plunger toward the distal end of the automatic injection device unobstructed by the first projection and the second projection.

20. A method of forming an automatic injection device to reduce the occurrence of a wet injection, the method comprising:
providing an automatic injection device having a distal end configured to deliver a medicament held in a container therein and a proximal end configured to be controllable by a user;
providing a housing having an inner surface defining a confined inner space of the automatic injection device, the housing having a length extending from the proximal end to the distal end along a longitudinal axis and a radial stop extending radially inward from a distal end of the inner surface;
providing a helical biasing member disposed in the confined inner space of the housing along the longitudinal axis having an inner bore and a length extending from a first terminal end of the helical biasing member to a second terminal end of the helical biasing member opposite the first terminal end, an inner diameter of the helical biasing member at a middle portion between the first terminal end and the second terminal end having a first inner diameter greater than a second inner diameter at the first terminal end or at the second terminal end;
providing a syringe plunger having a first end extending into the container and a second bifurcated end extending into the inner bore of the helical biasing member along the longitudinal axis, the bifurcated end having a first flexible arm and a second flexible arm, the first arm having a first projection at a first end thereof and the second arm having a second projection at a first end thereof, the first and the second flexible arms able to flex inwardly and outwardly relative to the longitudinal axis within the inner bore of the helical biasing member while maintaining an annular gap between the syringe plunger and the helical biasing member;
engaging the first projection and the second projection with the radial stop to maintain the syringe plunger in a latched position; and
providing a firing button including an inner ring configured to disengage the first projection and the second projection from the radial stop when the firing button is activated by the user.
